SEQUENCE LISTING (2 sequences)

SEQ ID NO: 1 - DNA, 22 bp, synthetic construct
  source 1..22: mol_type = other DNA
ccatccagcgtggtcgatagca

SEQ ID NO: 2 - DNA, 29 bp, synthetic construct
  source 1..29: mol_type = other DNA
caggtctcctgagaagtaagtgatgtggc

(19) United States
(12) Patent Application Publication
(10) Pub. No.: US 2024/0300934 A1
(43) Pub. Date: Sep. 12, 2024

(54) COMPOUNDS FOR MODULATING MYCOBACTERIUM TUBERCULOSIS RESPONSE

(71) Applicants: Trustees of Tufts College, Medford, MA (US); Rutgers, The State University of New Jersey, Piscataway, NJ (US)

(72) Inventors: Shumin Tan, Medford, MA (US); Anil Shelke, Piscataway, NJ (US); Jimmy S. Patel, Piscataway, NJ (US); Joel S. Freundlich, Piscataway, NJ (US); Matthew Sherwood, Piscataway, NJ (US); Samer Daher, Piscataway, NJ (US)

(21) Appl. No.: 18/573,813

(22) PCT Filed: Jul. 7, 2022
(86) PCT No.: PCT/US22/73519

§ 371 Date: Dec. 22, 2023

Related U.S. Application Data
(60) Provisional application No. 63/203,084, filed on Jul. 7, 2021.

(51) Int. Cl. C07D 413/12; A61K 31/437; A61K 31/506; C07D 263/58; C07D 471/04

(52) U.S. Cl. CPC C07D 413/12; A61K 31/437; A61K 31/506; C07D 263/58; C07D 471/04

(57) ABSTRACT

Disclosed herein are compounds and methods of using the same for treating a subject in need of a treatment for infection by a microbe and inhibiting growth or proliferation of a microbe. The method may comprise administering an effective amount of a compound or a pharmaceutical composition comprising the effective amount of a compound to the subject. Suitably the microbe is Mycobacterium tuberculosis.

### CROSS-REFERENCE TO RELATED APPLICATIONS

This application claims benefit of priority to U.S. Patent Application Ser. No. 63/203,084, filed Jul. 7, 2021, the contents of which is incorporated by reference in its entirety.

### STATEMENT REGARDING FEDERALLY SPONSORED RESEARCH

This invention was made with government support under grants AI114952 and AI143768 awarded by the National Institutes of Health. The government has certain rights in the invention.

### REFERENCE TO AN ELECTRONIC SEQUENCE LISTING

The contents of the electronic sequence listing (166118.01208.xml; Size: 90,379 bytes; and Date of Creation: Jul. 5, 2022) is herein incorporated by reference in its entirety.

### FIELD OF THE INVENTION

The disclosed technology is generally directed to compounds for modulating Mycobacterium tuberculosis environmental response, antitubercular drugs, and methods of use thereof.

### BACKGROUND OF THE INVENTION

Tuberculosis remains a major global public health issue, causing ˜1.4 million deaths a year. Drug resistance is an ever-increasing problem, reinforcing the need for new approaches in the targeting of Mycobacterium tuberculosis (Mtb) for therapeutic purposes. The local environments that Mtb encounters during establishment and continued chronic colonization of its host are highly varied, with differences arising in contexts ranging from lesion sublocations to immune-driven changes within a single host cell (Bhaskar et al., 2014; Harper et al., 2012; Huang et al., 2018b; Irwin et al., 2015; Lanoix et al., 2016b; Lenaerts et al., 2015; Mattila et al., 2013; Tan et al., 2013; Via et al., 2008). The ability of Mtb to recognize and adapt to local microenvironments is consequently critical for host colonization (Converse et al., 2009; Gautam et al., 2015; MacGilvary et al., 2019; Martin et al., 2006; Mehra et al., 2015; Perez et al., 2001), and a key microenvironment that Mtb must sense and respond to is that of the macrophage phagosome, which serves as a major replicative niche for Mtb (Abramovitch et al., 2011; Huang et al., 2018b; Pethe et al., 2004; Rohde et al., 2007; Tan et al., 2013). As a result, there is a need for compounds that modulate the response of Mtb to its environment and inhibit bacterial growth.

### BRIEF SUMMARY OF THE INVENTION

Disclosed herein are compounds and methods of using the same for treating a subject in need of a treatment for infection by a microbe and inhibiting growth or proliferation of a microbe.

One aspect of the technology provides for compounds. In some embodiments, the compound is a compound of Formula I

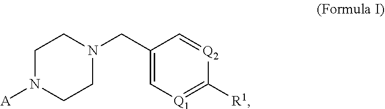

or a pharmaceutically acceptable salt thereof, wherein R¹ is —SR³, —OR³, or hydrogen and R³ is selected from an unsaturated or saturated, unbranched or branched, unsubstituted or substituted C₁-C₄ alkyl and hydrogen; Q₁ and Q₂ are independently selected from N or CH; and A is an unsubstituted or substituted heteroaryl. In some embodiments, A is an unsubstituted or substituted bicyclic heteroaryl. In some embodiments, the compound is a compound of Formula II

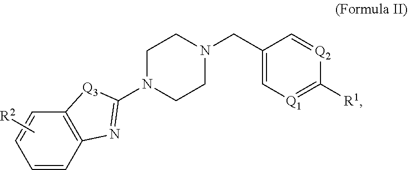

wherein Q₃ is O, S, or NH and R² is selected from hydrogen, cyano, a halo, an unsaturated or saturated, unbranched or branched, unsubstituted or substituted C₁-C₄ alkoxyl; or an unsaturated or saturated, unbranched or branched, unsubstituted or substituted C₁-C₄ alkyl.

In some embodiments, the compound is a compound of Formula III

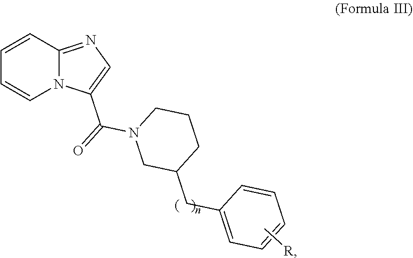

wherein n is an integer that equals to 0, 1, or 2; wherein the phenyl group is optionally substituted with one or more R substituents independently selected from a halo, a halo substituted alkyl, a halo substituted alkoxy, or nitrile.

Another aspect of the invention provides for a method for the treatment of a subject in need of a treatment for an infection by a microbe. The method may comprise administering an effective amount of a compound or a pharmaceutical composition comprising the effective amount of a compound to the subject. Suitably the microbe is Mycobacterium tuberculosis (Mtb).

Another aspect of the invention provides for a method for inhibiting growth or proliferation of a microbe in a host cell. The method may comprise contacting the host cell with an effective amount of a compound. In some embodiments, the host cell is a macrophage. Suitably the microbe is Mycobacterium tuberculosis (Mtb).

These and other aspects of the invention will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1D illustrate that the reporter-based chemical screen identifies compounds that modulate Mtb response to environmental chloride levels. (FIG. 1A) rv2390c′::GFP screen set-up. Schematic of the compound screen conducted in 384-well plates. DMSO-treated control wells with Mtb in 7H9, pH 6.4 media (negative, −) or 7H9, pH 6.4, 250 mM NaCl media (positive, +) were included in each plate. (FIG. 1B) Dual high [Cl⁻], slightly acidic pH conditions provide increased reporter dynamic range. Mtb carrying the rv2390c′::GFP reporter was grown for 6 days in pH 7 control media or in the indicated conditions in a 384-well plate format. Fold induction represents rv2390c′::GFP signal/OD₆₀₀ in each test condition as compared to the pH 7 control, measured by a microplate reader. Data are shown as means±SD from 16 wells. (FIGS. 1C and 1D) Dose response curve validation of two hit compounds. Mtb carrying the (FIG. 1C) rv2390c′::GFP reporter, or (FIG. 1D) rv1405c′::GFP reporter, was grown for 6 days in pH 6.4, 250 mM NaCl control media treated with the indicated compound, in a 384-well plate format with controls as shown in the schematic in (FIG. 1A). Normalized percent activation (NPA) was calculated by setting GFP signal/OD₆₀₀ of the reporter observed in the DMSO-treated positive control condition at 100%, and comparing the compound-treated GFP signal/OD₆₀₀ values to that baseline. Data are shown as means±SD from 2-3 wells.

FIGS. 2A-2E illustrate that small subset of compounds identified inhibit Mtb colonization of host macrophages. (FIG. 2A) Structures of compounds C5 and C6 and their respective analogs in the screen. (FIG. 2B) Dose response curve testing of effect of hit compounds on Mtb growth in J774 macrophage-like cells. Mtb constitutively expressing monomeric Kusabira Orange (mKO) was used to infect J774 cells in a 384-well format, and cells treated with compounds, 5 μM rifampicin (Rif), or DMSO as a carrier control. mKO fluorescence was measured 6 days post-infection with a microplate reader. The Rif samples were set as 100% inhibition of Mtb growth (versus that seen with DMSO treatment), and the mKO values in the compound-treated wells compared to that value to obtain the normalized percent inhibition (NPI). Data are shown as means±SD from 2 wells. (FIG. 2C) Compounds C5 and C6 increase rv2390c′::GFP reporter response upon bacterial exposure to high [Cl⁻]. Mtb carrying the rv2390c′::GFP reporter was grown for 6 days in 7H9, pH 7.0±250 mM NaCl or 7H9, pH 5.7, treated with DMSO as a carrier control, 10 μM C5 or 10 μM C6. Reporter induction in the 250 mM NaCl condition or the pH 5.7 condition was compared to that in the control 7H9, pH 7.0 condition for each treatment, with induction observed in the DMSO control set at 100%. GFP/OD₆₀₀ was calculated to allow normalization for bacterial numbers, with all measurements taken on a microplate reader. Data are shown as means±SD from 3-4 wells. (FIG. 2D) C5 and C6 inhibit growth in J774 cells. J774 macrophage-like cells were infected with Mtb constitutively expressing monomeric Kusabira Orange and treated with DMSO, 5 μM rifampicin, 10 μM C5, or 10 μM C6. Bacterial growth was tracked by fluorescence, with readings taken 6 days post-infection. DMSO is the carrier control, and growth in that condition is set at 100%. Data are shown as means±SD from 3-4 wells. p-values were obtained with a one-way ANOVA with a Dunnett's multiple corrections test, and treatment sets compared to the DMSO control. ****p<0.0001. (FIG. 2E) C5 and C6 inhibit growth in primary bone marrow-derived macrophages (BMDMs). BMDMs were infected with WT Mtb and bacterial load determined at indicated times. DMSO as a carrier control, 10 μM C5 or 10 μM C6 was added 2 hrs post-infection. Data are shown as means SD from 4 wells, pooled from two independent experiments.

FIGS. 3A-3H illustrate that structure-activity relationship analysis reveals that the chloride response phenotype of C6 tracks with inhibition of Mtb growth in macrophages. (FIG. 3A) Schematic for synthesis of C6. (FIG. 3B) Structures of C6 analogs synthesized. (FIGS. 3C-3E) C6 analogs that lose the ability to increase Mtb response to high [Cl⁻] also lose the Mtb growth inhibition phenotype in J774 cells and primary bone marrow-derived macrophages (BMDMs). (FIG. 3C) The rv2390c′::GFP reporter Mtb strain was grown in 7H9, pH 7±250 mM NaCl and treated with DMSO, 10 μM C6, or 10 μM C6 analogs (JSF-4271, JSF-4297, JSF-4298, or JSF-4300) for 9 days and samples fixed and reporter GFP induction analyzed by flow cytometry. Data are shown as means±SD from 3 independent experiments. p-values were obtained with a one-way ANOVA with a Dunnett's multiple corrections test, and treatment sets compared to the DMSO control. *p<0.05, **p<0.01. (FIG. 3D) J774 macrophage-like cells were infected with Mtb constitutively expressing monomeric Kusabira Orange and treated with DMSO, 5 μM rifampicin, 10 μM C6, or 10 μM C6 analogs (JSF-4271, JSF-4297, JSF-4298, or JSF-4300). Bacterial growth was tracked by fluorescence, with readings taken 6 days post-infection. DMSO is the carrier control, and growth in that condition is set at 100%. Data are shown as means±SD. from 4 wells. p-values were obtained with a one-way ANOVA with a Dunnett's multiple corrections test, and treatment sets compared to the DMSO control. ****p<0.0001. (FIG. 3E) BMDMs were infected with WT Mtb and bacterial load determined at indicated times. DMSO as a carrier control, 10 μM C6 or 10 μM C6, or 10 μM C6 analogs (JSF-4271, JSF-4297, JSF-4298, or JSF-4300) was added 2 hrs post-infection. Data are shown as means±SD from 3 wells. (FIG. 3F) C6 does not affect Mtb growth in standard 7H9 media. Mtb was grown in standard 7H9 media and cultures treated with DMSO or 10 μM of indicated compounds. OD₆₀₀ was tracked over time. Data are shown as means±SD from 3 independent experiments. (FIG. 3G) C6 analogs that lose the ability to increase Mtb response to high [Cl⁻] also lose the Mtb growth inhibition phenotype in cholesterol media. Mtb was grown in cholesterol media and cultures treated with DMSO or 10 μM of indicated compounds. OD₆₀₀ was tracked over time. Data are shown as means±SD from 3 independent experiments. (FIG. 3H) High [Cl⁻] in the presence of cholesterol media increases expression of the rv2390c′::GFP reporter. The rv2390c′::GFP reporter Mtb strain was grown in 7H9, pH 7±250 mM NaCl or cholesterol media, pH 7±250 mM NaCl, and samples fixed every 3 days for 12 days. Reporter GFP signal was analyzed by flow cytometry, and data are shown as means±SD from 3 independent experiments.

FIGS. 4A-4E illustrate that C6 does not alter phagosome maturation characteristics independent of infection. Carboxyfluorescein (FIG. 4A; pH readout), DQ-BSA/AF594 (FIG. 4B; proteolysis readout), or BAC/AF594 (FIG. 4C; [Cl⁻] readout) beads were added to murine bone marrow derived macrophages, treated with 10 μM C6 or DMSO as a carrier control, and fluorescence tracked over time with a microplate reader. Sensor beads were also added to wells containing only media, with no macrophages (“beads only, no cells”). Data are shown as means±SD from 3-5 wells, representative of 3 independent experiments. Comparison of carboxyfluorescein (FIG. 4D) and DQ-BSA/AF594 (FIG. 4E) readout for C6, JSF-4298, JSF-4271, JSF-4291, JSF-4299, and JSF-4300.

FIGS. 5A-5F illustrate that C6 accumulates in Mtb and host macrophages. (FIGS. 5A-5D) Intrabacterial accumulation of C6 is higher than the inactive analog JSF-4297. Mtb were grown in 7H9, pH 7±250 mM NaCl for 6 days, before 24 h exposure to C6 or JSF-4297, and analysis of intrabacterial compound content. (FIG. 5A) and (FIG. 5B) show dose-dependent intrabacterial accumulation of C6 versus JSF-4297 under control and high [Cl⁻] conditions respectively. (FIG. 5C) and (FIG. 5D) show the same data, but compare C6 or JSF-4297 accumulation under control versus high [Cl⁻] conditions in each case. (FIG. 5E) Intracellular accumulation of C6. J774 cells were exposed to indicated concentrations of C6 for 24 h, before analysis of the samples for intracellular compound content. (FIG. 5F) C6 accumulation during Mtb infection of J774 cells. J774 cells were infected with Mtb for 5 days, before treatment with 10 μM of indicated compound for 24 h, sample collection and analysis for total compound accumulation (within both J774 cells and bacteria within the J774 host cells). For (FIG. 5A)-(FIG. 5E), data are shown as means±SD from triplicate wells, representative of 2 independent experiments. In (FIG. 5F), data are shown as means±SD from triplicate wells, from 2-3 independent experiments. p-values were determined by one- (FIG. 5E) or two-way (FIGS. 5A-5D, FIG. 5F) ANOVA with Bonferroni post hoc test for all assays. ns p>0.05, *p<0.05, **p<0.01, ***p<0.001. The amount of accumulated compound as the number of moles was normalized by the cell number (Mtb or J774) prior to compound incubation.

FIGS. 6A-6D illustrate that C6 inhibits Mtb growth in vivo. (FIG. 6A and FIG. 6B) C6 inhibits Mtb growth in a short-term infection model. C3HeB/FeJ WT mice were infected with Mtb for 2 weeks (pre-treat), before mock-treatment or treatment with 250 mg/kg C6 (5 days/week via oral gavage) for a further 2 weeks. (FIG. 6A) shows CFUs from lung homogenates plated at 2 or 4 weeks post-infection. p-values were obtained with a Mann-Whitney statistical test. ** p<0.01. (FIG. 6B) shows lung pathology in the infected mice, with lung samples obtained from animals 2 weeks or 4 weeks post-infection, fixed, and processed for hematoxylin and eosin staining. Scale bar 200 m. (FIGS. 6C and 6D) C6 decreases Mtb load in a longer-term infection model. C3Heb/FeJ WT mice were infected with Mtb and infection allowed to establish for 6 weeks. Mice were then mock-treated or treated with 250 mg/kg C6 by oral gavage 5 days/week for 2 or 4 weeks (8 or 10 weeks total infection). Lungs were homogenized and plated for CFUs at indicated timepoints, shown in (FIG. 6C). p-values were determined by a Mann-Whitney statistical test on the mock versus circled C6-treated population. *p<0.05. (FIG. 6D) shows lung pathology in infected mice, with lung samples obtained from animals 6, 8 or 10 weeks post-infection, fixed, and processed for hematoxylin and eosin staining. Scale bar 200 m.

FIGS. 7A-7C illustrate accumulation of analogs JSF-4271 and JSF-4300 in Mtb and host macrophages. (FIG. 7A) Intrabacterial accumulation of the analogs JSF-4271 and JSF-4300 versus C6. Mtb were grown in 7H9, pH 7±250 mM NaCl for 6 days, before 24 h exposure to 10 M C6, JSF-4271, or JSF-4300, and analysis of intrabacterial compound content. (FIG. 7B) Intracellular accumulation of the analogs JSF-4271 and JSF-4300 versus C6. J774 cells were exposed to 10 μM C6, JSF-4271, or JSF-4300 for 24 h, before analysis of the samples for intracellular compound content. (FIG. 7C) Compound accumulation during Mtb infection of J774 cells. J774 cells were infected with Mtb for 5 days, before treatment with 10 μM of C6, JSF-4271, or JSF-4300 for 24 h, sample collection and analysis for total compound accumulation (within both J774 cells and bacteria within the J774 host cells). For (FIG. 7A)-(FIG. 7C), data are shown as means±SD from triplicate wells, representative of 2 independent experiments. p-values were determined by two-way (A) or one-way (FIGS. 7B and 7C) ANOVA with Bonferroni post hoc test for all assays. ns p>0.05, **p<0.01, ***p<0.001. The amount of accumulated compound as the number of moles was normalized by the cell number (Mtb or J774) prior to compound incubation.

FIG. 8 illustrates even chain fatty acids rescue inhibition of Mtb growth in cholesterol media by C6. Mtb was grown in cholesterol media ±4 mM acetate (added at the beginning of the experiment), or ±200 μM stearate (added every 3-4 days out to day 15), and cultures treated with DMSO or 10 μM C6. OD₆₀₀ was tracked over time. Data are shown as means±SD from 2 independent experiments.

FIG. 9 illustrates odd chain fatty acids, as well as vitamin B12, do not rescue inhibition of Mtb growth in cholesterol media by C6. Mtb was grown in cholesterol media or in cholesterol media with the indicated additional carbon source (all added at the beginning of the experiment, except for heptadecanoate, which was added at 200 μM every 3-4 days out to day 15), and cultures treated with DMSO or 10 μM C6. OD₆₀₀ was tracked over time. Data from day 18 of the assay is shown as means±SD from 2-4 independent experiments.

FIG. 10 illustrates Mtb response to cholesterol is inhibited by C6. qRT-PCR of gene expression of wild type Mtb exposed to cholesterol media, pH 7, treated with DMSO or 10 μM C6, for 4 hours. Data are shown as means±SD from 3 technical replicates. p-values were obtained with an unpaired t-test, **p<0.01, ****p<0.0001.

FIG. 11 illustrates JSF-4538 inhibits Mtb growth in vivo. C3HeB/FeJ WT mice were infected with Mtb for 2 or 6 weeks (pre-treatment), before mock-treatment or treatment with 150 mg/kg JSF-4538 (5 days/week via oral gavage) for a further 2 weeks. CFUs from lung homogenates plated at indicated times points post-infection are shown. p-value was obtained with a Mann-Whitney statistical test.

### DETAILED DESCRIPTION OF THE INVENTION

Disclosed herein are compounds for modulating response of a microbe such as Mycobacterium tuberculosis (Mtb) to its environment and method of using the same. As demonstrated by the Examples a reporter Mtb strain was exploited in a chemical screen for the discovery of compounds that modulate Mtb response to chloride (Cl⁻). Cl⁻ is an environmental cue closely linked to changes in pH, and Mtb responds synergistically to the simultaneous presence of acidic pH and high Cl⁻ levels. As also demonstrated in the Examples, the compounds disclosed herein modulate Mtb Cl⁻ response and inhibit bacterial growth both in macrophages and an in vivo model that recapitulates key lesion features seen during human infection. The compounds disclosed herein inhibit Mtb growth in cholesterol media but does not affect Mtb growth in rich broth culture and does not alter macrophage phagosome characteristics, emphasizing the significance of the infection context for its antitubercular effects. Structure-activity relationship studies indicate the importance of the 2-ethylthio group for the Cl⁻ response and macrophage inhibition phenotypes observed. Further, the intrabacterial, intracellular, and combined infection drug analysis approaches utilized here provide insight into the correlation between compound accumulation and antitubercular activity. These Examples reveal how accumulation of a compound within the bacterium or host cell affects activity, and how efficacy can be improved. Together, the Examples demonstrate the utility of the compounds disclosed herein to treat microbial infections.

Normal phagosome maturation entails acidification of the compartment, and while Mtb prevents complete maturation of the macrophage phagosome (Sturgill-Koszycki et al., 1996; Sturgill-Koszycki et al., 1994), the slight acidification that does occur continues to function as a major environmental cue for Mtb; almost half of the bacterial gene expression changes normally observed upon macrophage infection is lost if phagosome acidification is inhibited by concanamycin A treatment (Rohde et al., 2007). Chloride concentration ([Cl⁻]) within the phagosome is inversely related to pH, with [Cl⁻] increasing as pH decreases (Tan et al., 2013). Further, Mtb is able to respond to changes in environmental [Cl⁻], with the gene transcription changes observed in the presence of high external [Cl⁻] overlapping significantly with that seen upon bacterial exposure to acidic pH (Tan et al., 2013). Strikingly, the transcriptional response of Mtb to acidic pH and high [Cl⁻] is highly synergistic, reinforcing the role that Cl⁻ can play as an environmental cue for Mtb (Tan et al., 2013).

The ability of Mtb to respond to the ionic signals of pH and Cl⁻ is necessary for proper colonization of the host, with disruption of the two component regulatory system PhoPR, which is essential for pH response and important for Cl⁻ response (Abramovitch et al., 2011; Tan et al., 2013), leading to significant attenuation of Mtb growth in vivo (Martin et al., 2006; Perez et al., 2001). Other abundant ions, such as potassium (K⁺), also play an important role in Mtb host adaptation, as disruption of the Mtb Trk K⁺ uptake system results in a dampening of Mtb response to acidic pH and high [Cl⁻], and attenuation of Mtb growth in vivo (MacGilvary et al., 2019). The vital role of Mtb sensing and response to ionic cues for successful host colonization thus raises the possibility of exploiting this facet of Mtb-host interactions in therapeutic approaches. The synergistic response of Mtb to Cl⁻ and pH, and the heterogeneity of these environmental signals during in vivo infection, underscore the importance that Cl⁻ sensing and response can have for Mtb in the process of host colonization.

As further disclosed in the Examples, rv2390c′::GFP reporter Mtb strain was exploited in a chemical screen of 50,816 compounds to identify small molecule compounds that modulate Mtb response to Cl⁻. A tertiary screen examining the effect of hit compounds on Mtb growth in host macrophages, coupled with pharmacokinetic (PK) characterization, led to focusing of follow-up studies on the hit compound termed “C6” and related compounds. Structure-activity relationship (SAR) analysis identified the importance of the 2-ethylthio group in the ability of C6 to dysregulate Mtb Cl⁻ response and impair bacterial growth in host cells. C6 accumulates in both Mtb and host macrophages, but does not affect bacterial growth in standard rich broth or affect macrophage phagosome maturation characteristics. Mtb growth in cholesterol media was inhibited by treatment with C6, and rv2390c′::GFP reporter signal induction in response to high [Cl⁻] was intriguingly found to be increased in the context of cholesterol media, suggesting an intersection between Mtb metabolism and its response to Cl⁻. Most strikingly, oral administration of C6 in a C3HeB/FeJ murine infection model resulted in significant reduction in bacterial load, demonstrating the utility the utility of the compounds disclosed herein as antitubercular drugs.

### Compounds

One aspect of the invention provides for compounds, including chloride-response modulators. As used herein, a “chloride-response modulator” is a compound that is capable of modulating the response of a microbe, such as Mtb, to the amount or concentration of Cl⁻ in the environment surrounding the microbe. As used herein, “microbe” includes any microbe responsive to the compounds described herein. Suitably, microbes include microbes that grow or proliferate within host cells. Exemplary microbes include mycobacteria, such as Mtb and nontuberculous mycobacteria (NTM).

Chloride-response modulators as described herein, may be identified by the screening method disclosed in the Examples, such fluorescence-based screens utilizing reporter microbe strains. Chloride-response modulators may also be identified by other suitable screening methods. In the Examples, the threshold for identifying a chloride-response modulator was set at a response 3 standard deviations from a mean, but other screening thresholds may be utilized as well.

In some embodiments, the compounds described herein are capable of inhibiting the growth or proliferation of a microbe, such as Mtb. As demonstrated in the Examples, the compounds described herein are capable of inhibiting growth or proliferation of the microbe in vivo and in vitro.

In some embodiments, the compounds described herein are capable of inhibiting the growth or proliferation of a microbe in a host cell. In some embodiments, the compounds inhibit Mtb growth in host cells, such as macrophages, more than 20% at 20 μM. In particular embodiments, the compounds inhibit Mtb growth in host cells, such as macrophages, more than 30%, 40%, or 50% at 20 μM.

In some embodiments, the compounds described herein do not alter phagosome maturation. In particular embodiments, the compounds do not alter phagosomal acidification, proteolytic activity, or phagosomal maturation outside of physiologically acceptable ranges.

In some embodiments, the compounds described herein accumulate in microbes, such as Mtb.

In some embodiments, the compounds described herein accumulate in host cells, such as macrophages.

In some embodiments, the compounds described herein accumulate in microbe-infected host cells to a greater extent than uninfected host cells.

In some embodiments, the compounds described herein are capable of inhibiting the growth or proliferation of a microbe in a cholesterol media.

In some embodiments, the compounds described herein are capable of use in methods for treating a subject in need of a treatment for an infection by a microbe. As demonstrated in the examples, the compounds described herein inhibit microbe colonization in vivo. Accordingly, the compounds may be used in a treatment for tuberculosis or other microbial infection.

In some embodiments, the compounds described herein demonstrate physiochemical and pharmacokinetic properties suitable for use in methods for the treatment of a subject.

In some embodiments, the compounds have pharmaceutically acceptable solubility for preparing pharmaceutical composition. In particular embodiments, the compounds have aqueous solubility (S) in pH 7.4 phosphate buffered saline (PBS) greater than 5 μM. In other embodiments, the compounds have aqueous solubility greater than 10, 25, 50, or 75 μM.

In some embodiments, the compounds have pharmaceutically acceptable half-life (t_{1/2}). In particular embodiments, the compounds have a t_{1/2} greater than 1 min in mouse liver microsomes. In other embodiments, the compounds have a t_{1/2} greater than 5, 10, 15, 20, 25, 30, 35, or 45 min in mouse liver microsomes. In some embodiments, the compounds have a t_{1/2} between 1 and 100, 10 and 90, 20 and 80, 30 and 70, or 30 and 60 min in mouse liver microsomes.

In some embodiments, the compounds have pharmaceutically acceptable Area-under the curve (AUC). In particular embodiments, the compounds have an AUC_{0-5 h} greater than 25 h*ng/ml when a single 5 mg/kg oral (po) dose is administered to CD-1 mice. In other embodiments, the compounds have an AUC_{0-5 h} greater than 100, 500, 1000, 1500, 2000, 2500, 3000, 3500, 4000, 4500, or 5000 h*ng/ml when a single 5 mg/kg oral (po) dose is administered to CD-1 mice. In particular embodiments, the compounds have an AUC_{0-5 h} greater than 10,000 h*ng/ml when a single 25 mg/kg oral (po) dose is administered to CD-1 mice. In other embodiments, the compounds have an AUC_{0-5 h} greater than 20,000, 30,000, 40,000, 50,000, 60,000, 70,000, 80,000, or 90,000 h*ng/ml when a single 25 mg/kg oral (po) dose is administered to CD-1 mice.

In some embodiments, the compounds described herein are compounds of Formula I

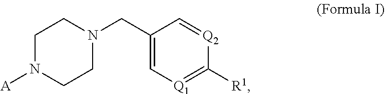

or a pharmaceutically acceptable salt thereof, where R¹ is —SR³, —OR³, or hydrogen; R³ is selected from an unsaturated or saturated, unbranched or branched, unsubstituted or substituted C₁-C₄ alkyl and hydrogen; Q₁ and Q₂ are independently selected from N or CH; and A is an unsubstituted or substituted heteroaryl. In some embodiments, A is an unsubstituted or substituted bicyclic heteroaryl.

In some embodiments, the compounds described herein are compounds of Formula II

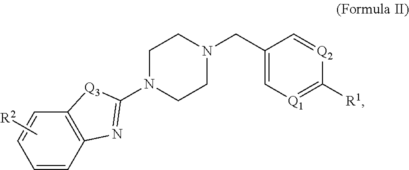

or a pharmaceutically acceptable salt thereof, where R¹ is —SR³, —OR³, or hydrogen; R³ is selected from an unsaturated or saturated, unbranched or branched, unsubstituted or substituted C₁-C₄ alkyl and hydrogen; Q₁ and Q₂ are independently selected from N or CH; Q₃ is O, S, or NH; and R² is selected from hydrogen, cyano, a halo, an unsaturated or saturated, unbranched or branched, unsubstituted or substituted C₁-C₄ alkoxyl; or an unsaturated or saturated, unbranched or branched, unsubstituted or substituted C₁-C₄ alkyl.

In some embodiments, A may be selected

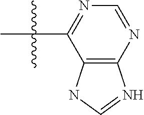 [SMILES: *c1ncnc2nc[nH]c12]

that may be optionally substituted.

In a particular embodiment, the compound is C6 analog.

In some embodiments, R¹ is —SR³. In other embodiments, R¹ is —OR³. In particular embodiments, R³ is methyl, ethyl, or propyl. In particular embodiments, R³ is ethyl, suitably —SCH₂CH₃.

In yet other embodiments, R³ is hydrogen.

In some embodiments, Q₁ and Q₂ are N. In other embodiments, Q₁ and Q₂ are CH. In yet other embodiments, one of Q₁ and Q₂ is N and the other is CH.

In some embodiments, Q₃ is O. In other embodiments, Q₃ is S. In yet other embodiments, Q₃ is NH.

In some embodiments, R² is hydrogen.

In some embodiments, the compound comprises are least one non-hydrogen substituent, R², that may be suitably positioned in the aromatic ring. In other embodiments, the compound comprises are least two non-hydrogen substituents, R², that may be suitably positioned in the aromatic ring. In embodiments having at least two non-hydrogen substituents, each R² may be independently selected from cyano, a halo, an unsaturated or saturated, unbranched or branched, unsubstituted or substituted C₁-C₄ alkoxyl; or an unsaturated or saturated, unbranched or branched, unsubstituted or substituted C₁-C₄ alkyl.

In some embodiments, R² is cyano.

In some embodiments, R² is a halo, such as F or Cl.

In some embodiments, R² is an unsaturated or saturated, unbranched or branched, unsubstituted or substituted C₁-C₄ alkoxyl, such as methoxy.

In some embodiments, R² is an unsaturated or saturated, unbranched or branched, unsubstituted or substituted C₁-C₄ alkyl, such as methyl or a halosubstituted methyl.

In particular embodiments, the compounds is selected from: JSF-4116 (C6); JSF-4298; JSF-4299; JSF-4300; JSF-4467; JSF-4471; JSF-4477; JSF-4507; JSF-4509; JSF-4516; JSF-4522; JSF-4525; JSF-4526; JSF-4527; JSF-4528; JSF-4534; JSF-4535; JSF-4538; JSF-4551; JSF-4562; JSF-4601; JSF-4602; JSF-4603; JSF-4604; JSF-4730; JSF-4747 and any combination thereof. In some embodiments, the compounds is any one of: JSF-4116 (C6); JSF-4298; JSF-4299; JSF-4300; JSF-4467; JSF-4471; JSF-4477; JSF-4507; JSF-4509; JSF-4516; JSF-4522; JSF-4525; JSF-4526; JSF-4527; JSF-4528; JSF-4534; JSF-4535; JSF-4538; JSF-4551; JSF-4562; JSF-4601; JSF-4602; JSF-4603; JSF-4604; JSF-4730; and JSF-4747. In some embodiments, the term compounds excludes one or more of JSF-4116 (C6); JSF-4298; JSF-4299; JSF-4300; JSF-4467; JSF-4471; JSF-4477; JSF-4507; JSF-4509; JSF-4516; JSF-4522; JSF-4525; JSF-4526; JSF-4527; JSF-4528; JSF-4534; JSF-4535; JSF-4538; JSF-4551; JSF-4562; JSF-4601; JSF-4602; JSF-4603; JSF-4604; JSF-4730; and JSF-4747 where indicated. In certain embodiments, the compounds is not JSF-4116 (C6) or C6 analog. Chemical structures and IUPAC names are provided in the Examples.

In some embodiments, the compounds described herein are compounds of Formula III

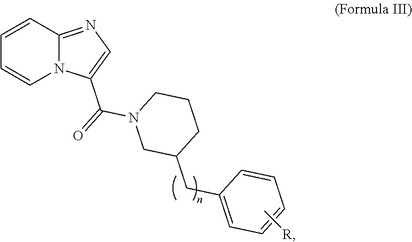

wherein n is an integer that equals to 0, 1, or 2; wherein the phenyl group is optionally substituted with one or more R substituents independently selected from a halo, a halo substituted alkyl, a halo substituted alkoxy, or nitrile.

In some embodiments, n is an integer that equals to 0. In some embodiments, n is an integer that equals to 1. In yet other embodiments, n is an integer that equals to 2.

In some embodiments, the phenyl group is not substituted.

In some embodiments, the phenyl group is substituted with one R substituent.

In some embodiments, the phenyl group is substituted with two R substituents. In some embodiments, the phenyl group is substituted with two of the same R substituents. In some embodiments, the phenyl group is substituted with two different R substituents.

In some embodiments, the phenyl group is substituted with more than two R substituents.

In some embodiments, R is a halo, such as F or Cl.

In some embodiments, R is a halo substituted alkyl such as a halo substituted methyl. In some embodiments, R is a fluoro substituted alkyl. In some embodiments, R is a trifluoromethyl.

In some embodiments, R is a halo substituted alkoxy such as a halo substituted methoxy.

In some embodiments, R is a fluoro substituted alkoxy. In some embodiments, R is a trifluoromethoxy.

In some embodiments, R is a nitrile.

In some embodiments, when n equals to 2, the phenyl group is not substituted at the meta position with a trifluoromethyl group. In certain embodiments, the compounds is not C5 or C5 analog.

The compounds of Formula I, Formula II, or Formula III may be prepared according to the representative synthesis scheme provided in the Examples and suitably modified for those of ordinary skill in the art.

The compounds described herein may include compounds that are not of Formula I, Formula II, or Formula III. In some embodiments, the compounds includes compounds identified as C6, C6 analog, C5, or C5 analog or any other compound having the properties of a chloride-response modulator described herein, such as those disclosed in Table 1.

As used herein, an asterisk “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number of ring carbon atoms. For example, the partially unsaturated carbocyclyl may contain 5-14, 5-12, 5-8, or 5-6 ring carbon atoms, and accordingly be referred to as a C₅-C₁₄, C₅-C₁₂, C₅-C₈, or C₅-C₆ membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

An “epoxide” is a cyclic ether with a three-atom ring typically include two carbon atoms and whose shape approximates an isosceles triangle. Epoxides can be formed by oxidation of a double bound where the carbon atoms of the double bond form an epoxide with an oxygen atom.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —R¹C(O)N(R²)—, —R¹C(O)N(R²)R³—, —C(O)NR²R³, or —C(O)NH₂, wherein R¹, R² and R³ are independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.)

### Pharmaceutical Compositions

The compounds disclosed herein may be formulated as pharmaceutical compositions that include: (a) an effective amount of one or more compounds; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods of disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the compounds or salts, esters, and/or amides, thereof. Solvate forms may include ethanol solvates, hydrates, and the like.

### Methods

Methods for treating subject with the compounds disclosed herein are provided. Suitably the method for treating in a subject comprises administering to the subject an effective amount of one or more of the compounds disclosed herein or a pharmaceutical composition comprising the effective amount of the one or more of the compounds disclosed herein. As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with one or more of the compounds disclosed herein. In some embodiments, the subject is responsive to therapy with one or more of the compounds disclosed herein in combination with one or more additional therapeutic agents. For example, a “subject in need of treatment” may include a subject in need of treatment for a microbial infection, such as Mtb infection. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

In some embodiments, the subject has tuberculosis. Subject's having tuberculosis may have active or latent Mtb infections and may have symptoms such as chronic cough, blood-containing mucus, fever, night sweats, or weight loss.

Methods for inhibiting growth or proliferation of a microbe are also provided. In some embodiments, administration of a compound to a subject or contacting a microbe or host cell with the compound provides for inhibiting growth or proliferation of the microbe.

In some embodiments, the methods described herein are practiced in vivo. In other embodiments, the methods described herein are practiced in vitro or ex vivo.

As used herein the term “effective amount” refers to the amount or dose of the compound, upon single or multiple dose administration to the subject, which provides the desired effect. In some embodiments, the effective amount is the amount or dose of the compound, upon single or multiple dose administration to the subject, which provides the desired effect in the subject under diagnosis or treatment. Suitably the desired effect may be inhibiting cellular infiltration by immune cells in the subject, inhibiting growth or proliferation of a microbe, accumulating the compound in the microbe or a host cell, modulating the response of the microbe to its local environment, including local Cl⁻ or pH environment, a physiochemical and pharmacokinetic properties suitable for use in methods for the treatment of a subject, not altering phagosome maturation, or any combination thereof.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Summary

Sensing and response to environmental cues, such as pH and chloride (Cl⁻), is critical in enabling Mycobacterium tuberculosis (Mtb) colonization of its host. Utilizing a fluorescent reporter Mtb strain in a chemical screen, compounds that dysregulate Mtb response to high Cl⁻ levels were identified. A subset of the hits also inhibited Mtb growth in host macrophages. Structure-activity relationship studies on the hit compound “C6” demonstrated a correlation between compound perturbation of Mtb Cl⁻ response and inhibition of bacterial growth in macrophages. C6 accumulated in both bacterial and host cells, and inhibited Mtb growth in cholesterol media, but not in rich media. Strikingly, oral administration of C6 was able to inhibit Mtb growth in vivo in a C3HeB/FeJ murine infection model. These Examples demonstrate how Mtb response to environmental cues can intersect with its metabolism and be exploited as antitubercular drugs.

### Representative Synthesis of C6 and Analogs Thereof

All reaction reagents and solvents were purchased from Sigma-Aldrich, Acros, Alfa Aesar, Tokyo Chemical Industry (TCI), or Fisher Scientific. Whenever required, reactions were conducted under a nitrogen atmosphere and anhydrous solvents were utilized. NMR spectra of the synthesized compounds were obtained on an Avance 500 MHz spectrometer from the Bruker Corporation. Mass spectral data were obtained at low resolution on an Agilent 6120 single quadrupole LC/MS system and at high-resolution on an Agilent 6220 accurate-mass time-of-flight system. All compounds for biological testing exhibited the expected NMR data, an LC purity at 250 nm>95%, and the expected mass spectral data. Reverse-phase high performance liquid chromatography (HPLC) and electrospray ionization (ESI) mass spectra were conducted on an Agilent 6120 single quadrupole LC/MS system using a reverse-phase EMID Millipore Chromolith SpeedRod RP-18e column (50×4.6 mm). Generally, a 10-100% gradient of acetonitrile/water containing 0.1% formic acid was implemented for the analysis of the samples. All compounds were purified to >95% peak area (i.e., purity) via an HPLC UV trace at 220 nm or 250 nm with observation of a low-resolution MS m z consistent with each compound. Purification of samples by flash chromatography was run on a Teledyne ISCO CombiFlash Rf+ system using a Teledyne RediSep normal phase silica gel column. For TLC, aluminum plates coated by silica gel 60 with F₂₅₄ fluorescent indicator from EMD Millipore were utilized. Preparative reverse-phase HPLC was conducted on a Varian (now Agilent) SD-1 preparative HPLC system equipped with an Agilent Pursuit (10 μm, 250×21.2 mm) C-18 column with detection UV wavelength set at 220 nm or 250 nm. A gradient of acetonitrile in water at a flow rate of 20 ml/min was used for the separation.

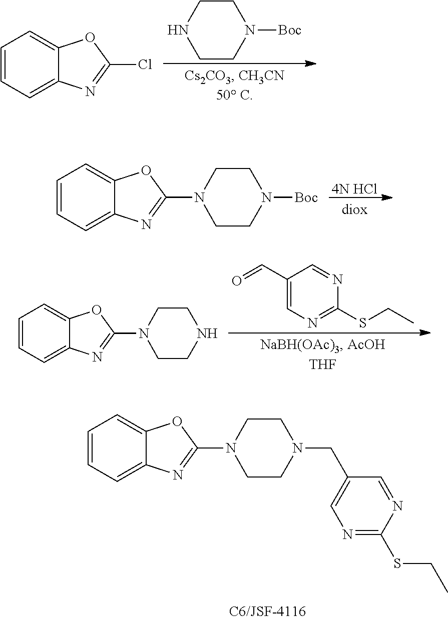

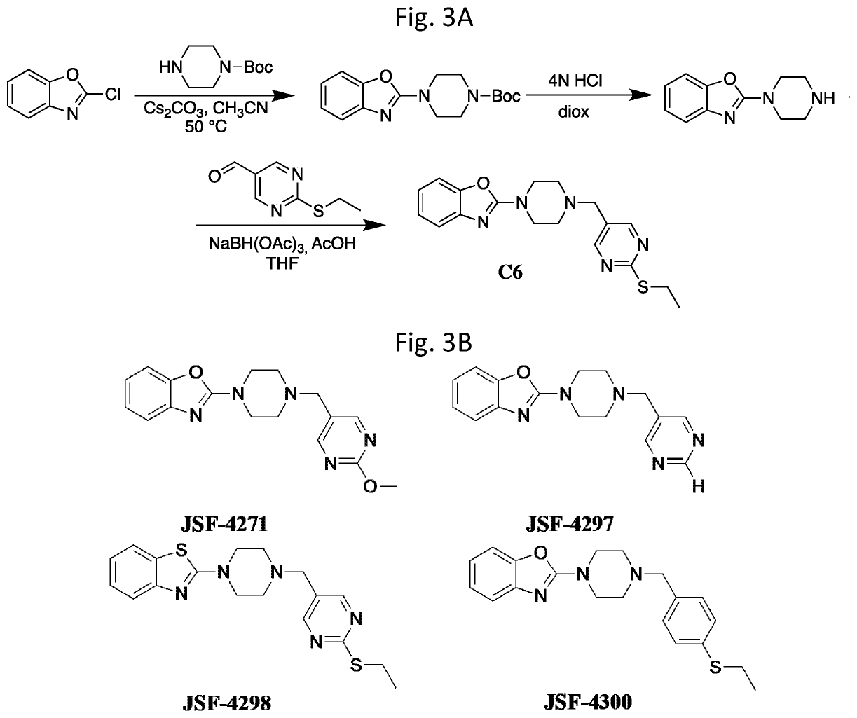

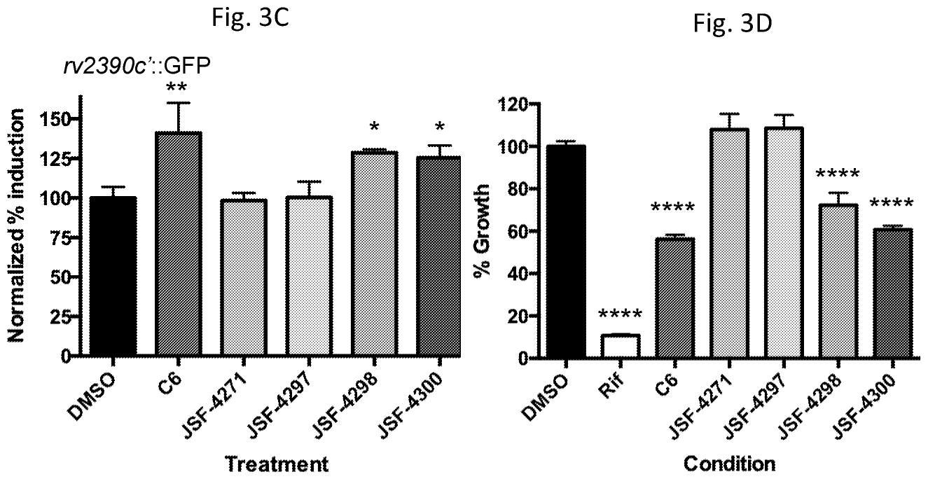

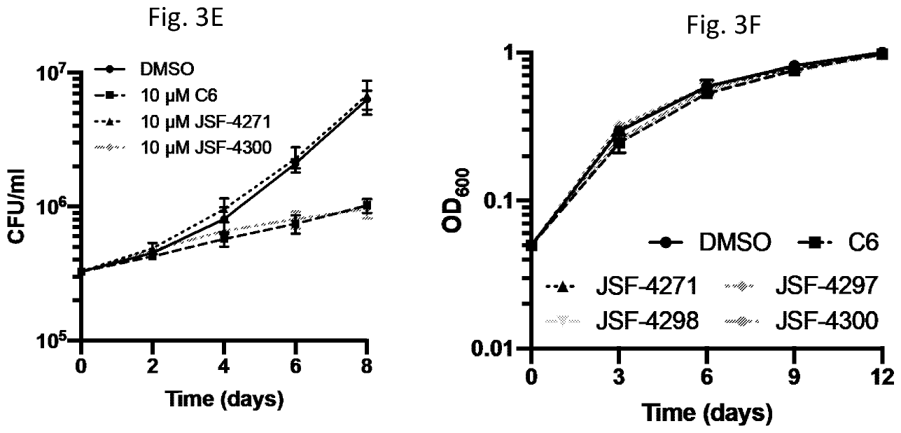

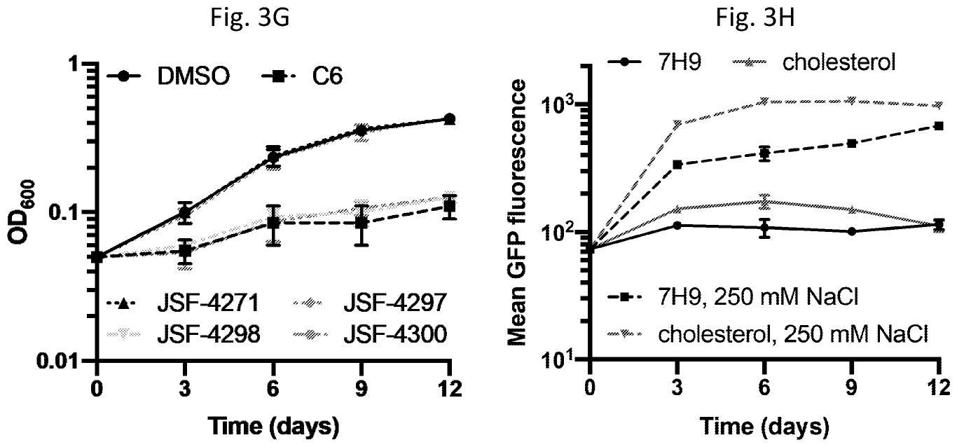

Synthesis of 2-(4-((2-(ethylthio)pyrimidin-5-yl)methyl)piperazin-1-yl)benzo[d]oxazole (C6) is shown in FIG. 3A. Tert-butyl 4-(benzo[d]oxazol-2-yl)piperazine-1-carboxylate. tert-butyl piperazine-1-carboxylate (2.23 g, 12.0 mmol, 1.2 eq) and cesium carbonate (3.58 g, 11.0 mmol, 1.1 eq) were diluted in 15 ml anhydrous acetonitrile at 50° C. To the stirred reaction mixture was added 2-chlorobenzo[d]oxazole (1.55 g, 10.0 mmol). The reaction was stirred at 50° C. for 12 h. The reaction mixture was then cooled to room temperature (rt) and solvent removed in vacuo. The resulting solid was diluted with water and extracted with ethyl acetate (3×50 ml). The combined organic layers were dried over anhydrous Na₂SO₄, and the solvent was removed in vacuo yielding the product as a white solid (3.00 g, 98.9%). H NMR (500 MHz, CDCl₃) 7.39 (d, J=5.0 Hz, 1), 7.28 (s, 1), 7.19 (t, J=7.5 Hz, 1), 7.05 (t, J=7.5 Hz, 1), 3.70 (br s, 4), 3.57 (br s, 4), 1.49 (s, 9). Calculated for C₁₆H₂₂N₃O₃ (M+H)⁺=304.2; Observed 304.2. This reaction product was used in the next step without purification. 2-(piperazin-1-yl)benzo[d]oxazole. To a round-bottom flask containing tert-butyl 4-(benzo[d]oxazol-2-yl)piperazine-1-carboxylate (3.00 g, 9.90 mmol) was added 4 N HCl in 1,4-dioxane (20 ml) dropwise at 0° C. The reaction was stirred at rt and monitored by TLC. Upon completion, the solvent was removed in vacuo, and the resulting solid was diluted with water, and taken to pH ˜8 with saturated sodium bicarbonate aqueous solution, and extracted with ethyl acetate (3×50 ml). The combined organic layers were dried over anhydrous Na₂SO₄, and the solvent was removed in vacuo yielding the product as an off-white solid (2.00 g, 99.4%): ¹H NMR (DMSO-d₆) 9.60 (br s, 1), 7.45 (d, J=5.0 Hz, 1), 7.35 (d, J=5.0 Hz, 1), 7.19 (t, J=7.5 Hz, 1), 7.05 (t, J=7.5 Hz, 1), 3.70 (t, J=4.7 Hz, 4), 3.24 (br s, 4). Also noted, 9.4 (br s), 5.7 (s, DCM), 3.6 (m), 3.3 (s, H₂O). Calculated for C₁₁H₁₄N₃O (M+H)⁺=204.1; Observed 204.0. This reaction product was used in the next step without purification. The final product (C6) was prepared via mixing 2-(piperazin-1-yl)benzo[d]oxazole (700 mg, 3.44 mmol), 2-(ethylthio)pyrimidine-5-carbaldehyde (579 mg, 3.44 mmol), and sodium triacetoxyborohydride (1.46 g, 6.88 mmol, 2.0 eq) dissolved in anhydrous tetrahydrofuran. Glacial acetic acid (0.2 ml) was added to the stirred mixture. The reaction was monitored by TLC until starting material was consumed. Upon completion, the pH was adjusted to ˜9 with 1 M NaOH_{(aq)}, and extracted with diethyl ether (3×100 ml). The combined organic layers were washed with saturated aqueous brine solution, dried over anhydrous Na₂SO₄, and the solvent was removed in vacuo. The crude product were purified by silica gel flash column chromatography with EtOAc/hexanes to afford the product as white solid (0.750 g, 61.3%): ¹H NMR (500 MHz, CDCl₃) δ 8.51 (br s, 2), 7.32 (d, J=7.9 Hz, 1), 7.25 (d, J=7.8 Hz, 1), 7.18 (t, J=7.6 Hz, 1), 7.03 (t, J=7.9 Hz, 1), 3.75 (br s, 4), 3.52 (br s, 2), 3.16 (q, J=7.3 Hz, 2), 2.61 (br s, 4), 1.41 (t, J=7.3 Hz, 3). ¹³C NMR (125 MHz, DMSO) δ 169.9, 161.8, 158.2 (2 carbons), 148.3, 142.9, 126.3, 124.0 (2 carbons), 120.6, 115.9, 108.9, 56.0, 51.5 (2 carbons), 45.2, 24.5, 14.6. Calculated for C₁₈H₂₁N₅OS (M+H)⁺=356.1567; Observed 356.1541.

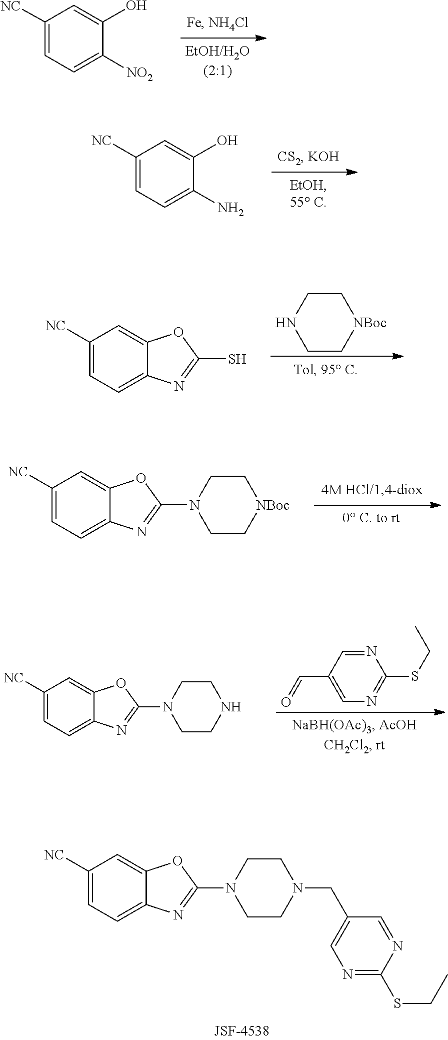

Synthesis of 2-(4-((2-(ethylthio)pyrimidin-5-yl)methyl)piperazin-1-yl)benzo[d]oxazole-6-carbonitrile (JSF-4538): Preparation of 4-amino-3-hydroxybenzonitrile: To a vial was added a solution of 3-hydroxy-4-nitrobenzonitrile (242 mg, 1.47 mmol), Fe (659 mg, 11.8 mmol, 8.0 eq), and NH₄Cl (314 mg, 5.88 mmol, 4.0 eq) in 2:1 mixture of EtOH/H₂O (9 mL). The reaction was stirred at 55° C. for 5 h. Upon confirming the completion of reaction via LC-MS, the reaction mixture was allowed to cool to rt and passed through a pad of Celite, which was then washed with EtOAc (20 mL). The majority of solvent was removed in vacuo to give a yellow liquid. The crude product was purified by silica gel flash column chromatography using a gradient of 10% MeOH in CH₂Cl₂ to give the desired product as a yellow solid (178 mg, 1.33 mmol, 90%). Calculated for C₇H₇N₂O (M+H)⁺=135.0; observed 135.0.

Preparation of 2-mercaptobenzo[d]oxazole-6-carbonitrile: To a 50 mL round-bottom flask was added a solution of 4-amino-3-hydroxybenzonitrile (178 mg, 1.33 mmol) and potassium hydroxide (223 mg, 3.98 mmol, 3.0 eq) in 5 mL EtOH. Then carbon disulfide (0.302 mg, 0.234 mL, 3.98 mmol, 3.0 eq) was added, and the mixture was stirred at 55° C. for 14 h. Upon confirming the completion of reaction via LC-MS, the reaction mixture was allowed to cool to rt and solvent was removed under reduced pressure to afford a crude residue. The residue was acidified with a few drops of 1 N HCl_{(aq)} until providing a pH ˜2 solution. The product was then extracted with EtOAc (3×10 mL). The combined organic layers were washed successively with saturated aqueous brine solution, dried over anhydrous sodium sulfate, filtered, and concentrated in vacuo. The yellow solid obtained was used immediately in the next step without further purification (230 mg, 1.31 mmol, 98%). Calculated for C₈H₅N₂OS (M+H)⁺=177.0; observed 177.0.

Preparation of tert-butyl 4-(6-cyanobenzo[d]oxazol-2-yl)piperazine-1-carboxylate: To a 100 mL round-bottom flask was added a solution 2-mercaptobenzo[d]oxazole-6-carbonitrile (230 mg, 1.31 mmol) and tert-butyl piperazine-1-carboxylate (488 mg, 2.62 mmol, 2.0 eq) in 12 mL toluene. The mixture was stirred at 95° C. for 5 h. Upon confirming the completion of reaction via LC-MS, the reaction mixture was allowed to cool to rt and solvent was removed under reduced pressure to give crude product as beige solid. The crude product was purified by silica gel flash column chromatography using a gradient of 50-70% EtOAc in hexanes to give the desired product as a white solid (378 mg, 1.15 mmol, 88%). Calculated for C₁₇H₂N₄O₃ (M+H)⁺=329.2; observed 329.2.

Preparation of 2-(piperazin-1-yl)benzo[d]oxazole-6-carbonitrile: To a 50 mL round-bottom flask was added a solution tert-butyl 4-(6-cyanobenzo[d]oxazol-2-yl)piperazine-1-carboxylate (378 mg, 1.15 mmol) in 12 mL 4 M HCl/1,4-dioxane. The mixture was stirred at 0° C. and warmed gradually to rt for 2 h. Upon confirming completion of the reaction via LC-MS, the solvent was removed under reduced pressure to give a crude residue. The residue was treated with 25 mL of saturated bicarbonate solution until providing a pH ˜8 solution. The product was then extracted with EtOAc (3×20 mL). The combined organic layers were washed successively with saturated aqueous brine solution, dried over anhydrous sodium sulfate, filtered and concentrated in vacuo. The white solid obtained was used immediately in the next step without further purification (250 mg, 1.09 mmol, 95%). Calculated for C₁₂H₁₃N₄O (M+H)⁺=229.1; observed 229.0.

Preparation of 2-(4-((2-(ethylthio)pyrimidin-5-yl)methyl)piperazin-1-yl)benzo[d]oxazole-6-carbonitrile (JSF-4538): To a 50 mL round-bottom flask was added a solution of 2-(ethylthio)pyrimidine-5-carbaldehyde (184 mg, 1.09 mmol) and 2-(piperazin-1-yl)benzo[d]oxazole-6-carbonitrile (250 mg, 1.09 mmol, 1.0 eq) in 6 mL CH₂Cl₂. Then NaBH₃CN (462 mg, 2.18 mmol, 2.0 eq) was added followed by AcOH (0.075 mL, 1.1 mmol, 1.0 eq) and the mixture was stirred at rt for 10 h. Upon confirming the completion of the reaction via LC-MS, the reaction mixture was diluted with water (10 mL), and extracted with CH₂Cl₂ (3×10 mL). The combined organic layers were washed successively with saturated aqueous brine solution, dried over anhydrous sodium sulfate, filtered, and concentrated in vacuo to afford a beige solid. The crude reaction product was purified by silica gel flash chromatography using a gradient of 90% EtOAc in hexanes to give the desired product as white solid (172 mg, 0.453 mmol, 42%): ¹H NMR (500 MHz, CD₃CN) δ 8.50 (s, 2), 7.62 (d, J=1.3 Hz, 1), 7.51 (dd, J=8.2, 1.5 Hz, 1), 7.31 (d, J=8.1 Hz, 1), 3.68 (m, 4), 3.51 (s, 2), 3.13 (q, J=7.3 Hz, 2), 2.55 (m, 4), 1.36 (t, J=7.3 Hz, 3). Also noted 2.1 (s, H₂O). Calculated for C₁₉H₂₁N₆OS (M+H)⁺=381.1499; observed 381.1496.

### Representative Synthesis of C5 and Analogs Thereof

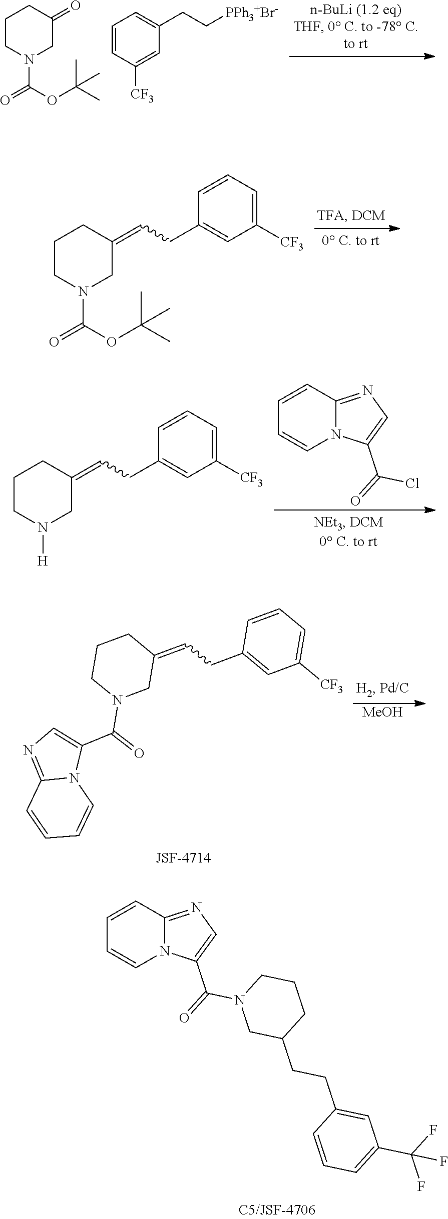

Synthesis of imidazo[1,2-a]pyridin-3-yl(3-(2-(3-(trifluoromethyl)phenyl)ethylidene)piperidin-1-yl)methanone (JSF-4714). tert-butyl 3-(2-(3-(trifluoromethyl)phenyl)ethylidene)piperidine-1-carboxylate: A round-bottom flask was loaded with the bromotriphenyl(3-(trifluoromethyl)phenethyl)-λ⁵-phosphane (6.4 g, 12 mmol, 1.2 equiv) and tetrahydrofuran (30 mL). After cooling to 0° C., n-BuLi (5.0 mL, 12 mmol, 2.5 M in hexanes) was added dropwise and the mixture was stirred at 0° C. for 20 min. After cooling to −78° C., tert-butyl 3-oxopiperidine-1-carboxylate (2.0 g, 10 mmol, 1.0 equiv) dissolved in THF (15 mL) was added dropwise. The mixture was then allowed to warm to rt and was stirred for overnight. The reaction was quenched with a saturated solution of NH₄Cl (30 mL) and the aqueous layer was extracted with diethyl ether (3×30 mL). The combined organic layers were dried over anhydrous Na₂SO₄, filtered, and concentrated under reduced pressure. The residue was finally purified by flash column chromatography (0-40% hexanes/EtOAc) on silica gel to afford the title compound as a colorless liquid (2.6 g, 7.3 mmol, 74%): ¹H NMR (500 MHz, CDCl₃) δ 7.44 (dd, J=9.0, 4.0 Hz, 2), 7.38 (m, 2), 5.34 (t, J=7.5 Hz, 1), 4.06 (s, 2), 3.51-3.48 (m, 4), 2.27 (t, J=6.1 Hz, 2), 1.66-1.62 (m, 2), 1.44 (s, 9); (trans:cis 3:1), HRMS Calculated for C₁₉H₂₄F₃NO₂Na [M+Na]⁺=378.1657, observed 378.1646.

Imidazo[1,2-a]pyridin-3-yl(3-(2-(3-(trifluoromethyl)phenyl)ethylidene)piperidin-1-yl)methanone (JSF-4714): tert-butyl 3-(2-(3-(trifluoromethyl)phenyl)ethylidene)piperidine-1-carboxylate (930 mg, 2.61 mmol) was dissolved in 60 mL of dry CH₂Cl₂ at 0° C. and treated with 3 mL of trifluoroacetic acid (TFA). The resulting solution was warmed to rt and stirred for 2 h. The solvents were removed in vacuo. The crude salt was then dissolved in toluene, concentrated, and dried in vacuo to remove residual TFA. The crude TFA salt was then dissolved in 40 mL of dry CH₂Cl₂ and cooled to 0° C. Triethylamine (1.09 mL, 7.85 mmol, 3.00 equiv) was added to the reaction mixture and stirred for 10 min at 0° C. Then, imidazo[1,2-a]pyridine-3-carbonyl chloride (567 mg, 3.14 mmol, 1.20 equiv) was added slowly to the reaction mixture and the resulting solution was warmed to rt and stirred overnight. After completion of the reaction as judged by TLC, the mixture was concentrated in vacuo. The crude reaction mixture was then purified by flash column chromatography (0-100% hexanes/EtOAc) to afford the title compound as a white sticky solid (460 mg, 1.15 mmol, 44.0% over the 2 steps): ¹H NMR (500 MHz, CDCl₃) δ 8.98 (d, J=7.0 Hz, 1), 7.89 (s, 1), 7.71 (m, 1), 7.41 (dd, J=15.8, 7.7 Hz, 3), 7.35-7.28 (m, 2), 6.97 (m, 1), 5.46 (t, J=7.5 Hz, 1), 4.47 (s, 2), 3.93 (m, 2), 3.51 (d, J=7.0 Hz, 2), 2.43 (t, J=6.1 Hz, 2), 1.80 (m, 2), Also noted δ 1.2 (br s), 0.9 (m). HRMS Calculated for C₂₂H₂₁F₃N₃O [M+H]⁺=400.1637, observed 400.1632.

Synthesis of imidazo[1,2-a]pyridin-3-yl(3-(3-(trifluoromethyl)phenethyl)piperidin-1-yl)methanone (JSF-4706/C5): To a suspension of imidazo[1,2-a]pyridin-3-yl(3-(2-(3-(trifluoromethyl)phenyl)ethylidene)piperidin-1-yl)methanone (100 mg, 0.250 mmol) in methanol (5 mL) at rt was added 10 wt % palladium on carbon (0.015 g). The resulting reaction mixture was stirred for 12 h at rt under a hydrogen-filled balloon (1 atm). After completion of the reaction, the reaction mixture was filtered through a pad of Celite and the solvent was removed under reduced pressure. The residue was finally purified by flash column chromatography (0-100% hexanes/EtOAc) on silica gel to afford the desired compound as a white solid (0.026 g, 0.064 mmol, 26%): ¹H NMR (500 MHz, CDCl₃) δ 9.06 (d, J=6.9 Hz, 1), 8.07 (d, J=8.8 Hz, 1), 7.98 (s, 1), 7.68 (t, J=7.7 Hz, 1), 7.44 (d, J=8.5 Hz, 2), 7.37 (dd, J=12.9, 7.7 Hz, 2), 7.23 (t, J=7.0 Hz, 1), 4.47-4.33 (m, 2), 3.18 (s, 1), 2.87 (d, J=10.5 Hz, 1), 2.74 (d, J=8.8 Hz, 2), 2.04 (d, J=13.1 Hz, 1), 1.86 (d, J=16.4 Hz, 1), 1.68-1.58 (m, 3), 0.89-0.82 (m, 2), Also noted δ 1.25 (br s, grease). HRMS Calculated for C₂₂H₂₂F₃N₃O₃[M+H]+=402.1793, observed 402.1786.

### Exemplary Compounds

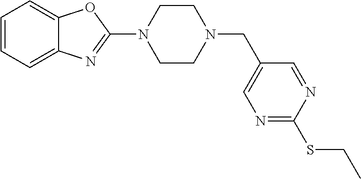 [SMILES: CCSc1ncc(CN2CCN(c3nc4ccccc4o3)CC2)cn1]

2-(4-((2-(ethylthio)pyrimidin-5-yl)methyl)piperazin-1-yl)benzo[d]oxazole (C6 or JSF-4116): ¹H NMR (500 MHz, CDCl₃) δ 8.51 (br s, 2), 7.32 (d, J=7.9 Hz, 1), 7.25 (d, J=7.8 Hz, 1), 7.18 (t, J=7.6 Hz, 1), 7.03 (t, J=7.9 Hz, 1), 3.75 (br s, 4), 3.52 (br s, 2), 3.16 (q, J=7.3 Hz, 2), 2.61 (br s, 4), 1.41 (t, J=7.3 Hz, 3). ¹³C NMR (125 MHz, DMSO) δ 169.9, 161.8, 158.2 (2 carbons), 148.3, 142.9, 126.3, 124.0 (2 carbons), 120.6, 115.9, 108.9, 56.0, 51.5 (2 carbons), 45.2, 24.5, 14.6. Calculated for C₁₈H₂₁N₅OS (M+H)⁺=356.1567; Observed 356.1541.

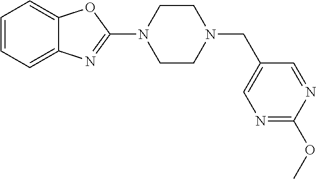 [SMILES: COc1ncc(CN2CCN(c3nc4ccccc4o3)CC2)cn1]

2-(4-((2-methoxypyrimidin-5-yl)methyl)piperazin-1-yl)benzo[d]oxazole (JSF-4271): ¹H NMR (500 MHz, acetone-d₆) δ 8.53 (s, 2), 7.31 (d, J=7.9 Hz, 1), 7.26 (d, J=7.8 Hz, 1), 7.14 (t, J=7.6 Hz, 1), 7.01 (t, J=7.7 Hz, 1), 3.95 (s, 3), 3.70-3.67 (m, 4), 3.57 (s, 2), 2.61-2.57 (m, 4). ¹³C NMR (125 MHz, acetone-d₆) δ 166.4, 163.2, 160.8 (2 carbons), 149.9, 144.6, 125.6, 124.8, 121.4, 117.1, 109.6, 57.4, 55.1, 52.8 (2 carbons), 46.5 (2 carbons). Calculated for C₁₇H₁₉N₅O₂ (M+H)⁺=326.1638; Observed 326.1611.

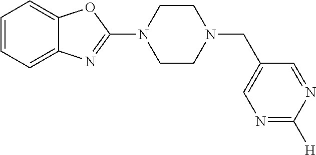 [SMILES: c1ccc2oc(N3CCN(Cc4cncnc4)CC3)nc2c1]

2-(4-(pyrimidin-5-ylmethyl)piperazin-1-yl)benzo[d]oxazole (JSF-4297): ¹H NMR (500 MHz, DMSO-d₆) δ 9.12 (s, 1), 8.77 (s, 2), 7.39 (d, J=7.9 Hz, 1), 7.30-7.27 (m, 1), 7.14 (td, J=7.6, 1.0 Hz, 1), 7.01 (td, J=7.8, 1.2 Hz, 1), 3.61 (s, 4), 3.35 (s, 2), 2.54-2.51 (m, 4). ¹³C NMR (125 MHz, acetone-d₆) δ 163.2, 158.6, 158.3 (2 carbons), 149.9, 144.6, 132.7, 124.8, 121.4, 117.1, 109.6, 58.0, 53.0 (2 carbons), 46.5 (2 carbons). Calculated for C₁₆H₁₇N₅O (M+H)⁺=296.1533; Observed 296.1504.

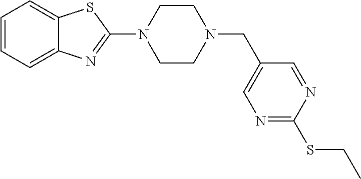 [SMILES: CCSc1ncc(CN2CCN(c3nc4ccccc4s3)CC2)cn1]

2-(4-((2-(ethylthio)pyrimidin-5-yl)methyl)piperazin-1-yl)benzo[d]thiazole (JSF-4298) ¹H NMR (500 MHz, acetone-d₆) δ 8.57 (s, 2), 7.70 (d, J=7.8 Hz, 1), 7.46 (d, J=8.1 Hz, 1), 7.28 (t, J=7.7 Hz, 1), 7.07 (t, J=7.6 Hz, 1), 3.66-3.63 (m, 4), 3.59 (s, 2), 3.14 (q, J=7.3 Hz, 2), 2.64-2.61 (m, 4), 1.36 (t, J=7.3 Hz, 3). ¹³C NMR (125 MHz, acetone-d₆) δ 171.8, 169.3, 159.0 (2 carbons), 154.1, 132.0, 127.6, 126.9 (2 carbons), 122.3, 121.8, 119.9, 57.6, 53.0 (2 carbons), 49.3, 25.6, 15.1. Calculated for C₁₈H₂₁N₅S₂ (M+H)⁺=372.1338; Observed 372.1310.

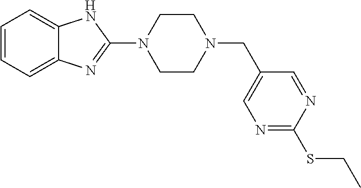

2-(4-((2-(ethylthio)pyrimidin-5-yl)methyl)piperazin-1-yl)-1H-benzo[d]imidazole (JSF-4299): ¹H NMR (500 MHz, CD₃CN) δ 8.53 (s, 2), 7.24 (d, J=3.4 Hz, 2), 7.03-6.98 (m, 2), 3.54-3.46 (m, 7), 3.16 (q, J=6.2 Hz, 2), 2.59 (t, 4), 1.39 (t, J=6.7 Hz, 3). Also noted 7.3 (m), 4.1 (q, EtOAc), 3.7 (s), 2.2 (br s, H₂O), 2.1 (s, EtOAc), 1.2 (t, EtOAc).

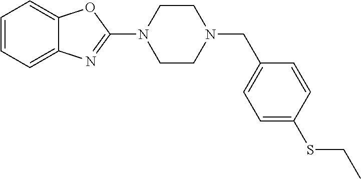 [SMILES: CCSc1ccc(CN2CCN(c3nc4ccccc4o3)CC2)cc1]

2-(4-(4-(ethylthio)benzyl)piperazin-1-yl)benzo[d]oxazole (JSF-4300): ¹H NMR (500 MHz, acetone-d₆) δ 7.32 (s, 4), 7.30 (s, 1), 7.26 (d, J=7.8 Hz, 1), 7.14 (td, J=7.7, 0.9 Hz, 1), 7.01 (td, J=7.9, 1.1 Hz, 1), 3.69-3.66 (m, 4), 3.54 (s, 2), 2.99-2.95 (m, 2), 2.57-2.54 (m, 4), 1.28 (t, J=7.3 Hz, 3). ¹³C NMR (125 MHz, acetone-d₆) δ 163.3, 150.0, 144.7, 137.0, 136.4, 130.6 (2 carbons), 129.6 (2 carbons), 124.8, 121.4, 117.1, 109.6, 63.0, 53.1 (2 carbons), 46.6 (2 carbons), 27.9, 14.9. Calculated for C₂₀H₂₃N₃OS (M+H)⁺=354.1662; Observed=354.1634.

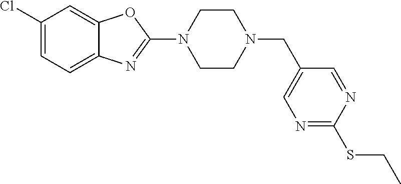 [SMILES: CCSc1ncc(CN2CCN(c3nc4ccc(Cl)cc4o3)CC2)cn1]

6-chloro-2-(4-((2-(ethylthio)pyrimidin-5-yl)methyl)piperazin-1-yl)benzo[d]oxazole (JSF-4467): ¹H NMR (500 MHz, d₆-DMSO) δ 8.71 (s, 2), 7.64 (d, J=1.8 Hz, 1), 7.34 (d, J=8.4 Hz, 1), 7.25 (dd, J=8.4, 1.9 Hz, 1), 4.30 (s, 2), 3.83 (br s, 4), 3.30 (br s, 4), 3.14 (q, J=7.3 Hz, 2), 1.34 (t, J=7.0 Hz, 3). Also noted, 10.0 (s), 9.0 (s), 4.0 (br s).

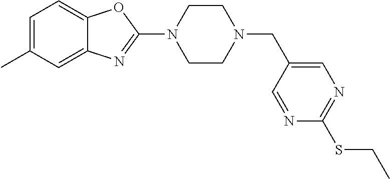 [SMILES: CCSc1ncc(CN2CCN(c3nc4cc(C)ccc4o3)CC2)cn1]

2-(4-((2-(ethylthio)pyrimidin-5-yl)methyl)piperazin-1-yl)-5-methylbenzo[d]oxazole (JSF-4471): ¹H NMR (500 MHz, CDCl₃) δ 8.64 (br s, 2), 7.08 (d, J=8.2 Hz, 1), 6.93 (d, J=8.3 Hz, 1), 6.89 (s, 1), 4.21 (br s, 4), 3.42 (br s, 4), 3.18 (q, J=7.0 Hz, 2), 2.42 (s, 2), 2.38 (s, 3), 1.40 (t, J=6.6 Hz, 3). Also noted 6.3 (br s), 5.3 (s, DCM), 2.1 (s).

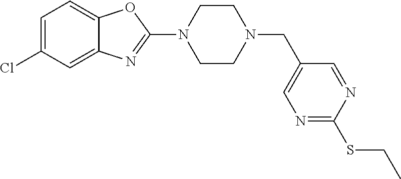 [SMILES: CCSc1ncc(CN2CCN(c3nc4cc(Cl)ccc4o3)CC2)cn1]

5-chloro-2-(4-((2-(ethylthio)pyrimidin-5-yl)methyl)piperazin-1-yl)benzo[d]oxazole (JSF-4477): ¹H NMR (500 MHz, CDCl₃) δ 8.50 (br s, 2), 7.31 (d, J=1.9 Hz, 1), 7.14 (d, J=8.5 Hz, 1), 6.99 (d, J=7.2 Hz, 1), 3.71 (br s, 4), 3.50 (br s, 2), 3.17 (q, J=7.4 Hz, 2), 2.58 (br s, 4), 1.41 (t, J=7.4 Hz, 3). Also noted 5.3 (s, DCM), 5.1 (s), 4.1 (q, EtOAc), 3.6 (s), 2.0 (s, EtOAc), 1.5 (s, H₂O), 1.2 (m, EtOAc).

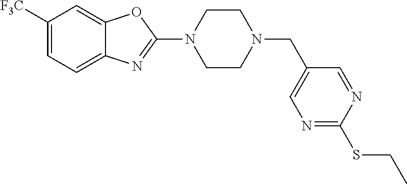 [SMILES: CCSc1ncc(CN2CCN(c3nc4ccc(C(F)(F)F)cc4o3)CC2)cn1]

2-(4-((2-(ethylthio)pyrimidin-5-yl)methyl)piperazin-1-yl)-6-(trifluoromethyl)benzo[d]oxazole (JSF-4507): ¹H NMR (500 MHz, CDCl₃) δ 8.54 (s, 2), 7.51 (s, 1), 7.47 (d, J=8.3 Hz, 1), 7.39 (d, J=8.1 Hz, 1), 3.79 (br s, 4), 3.54 (br s, 2), 3.17 (q, J=7.4 Hz, 2), 2.64 (br s, 4), 1.41 (t, J=7.4 Hz, 3). Also noted 5.1 (s), 3.2 (s) and 1.6 (s, H₂O). ¹⁹F NMR (470 MHz, CDCl₃) δ −60.6 (s).

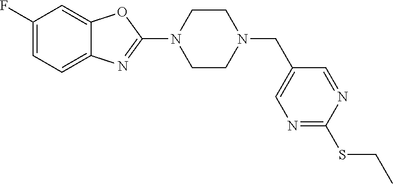 [SMILES: CCSc1ncc(CN2CCN(c3nc4ccc(F)cc4o3)CC2)cn1]

2-(4-((2-(ethylthio)pyrimidin-5-yl)methyl)piperazin-1-yl)-6-fluorobenzo[d]oxazole (JSF-4509): ¹H NMR (500 MHz, CD₃CN) δ 8.64 (s, 2), 7.28 (dd, J=8.6, 4.9 Hz, 1), 7.20 (dd, J=8.3, 2.3 Hz, 1), 7.01-6.96 (m, 1), 4.21 (s, 2), 3.92 (s, 4), 3.32 (s, 4), 3.15 (q, J=7.3 Hz, 2), 1.36 (t, J=7.3 Hz, 3). Also noted 1.3 (d).

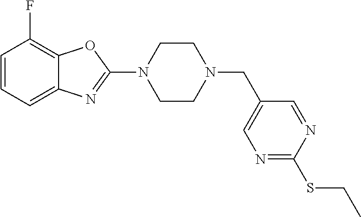 [SMILES: CCSc1ncc(CN2CCN(c3nc4cccc(F)c4o3)CC2)cn1]

2-(4-((2-(ethylthio)pyrimidin-5-yl)methyl)piperazin-1-yl)-7-fluorobenzo[d]oxazole (JSF-4516): H NMR (500 MHz, CDCl₃) δ 8.59 (s, 2), 7.09-7.15 (m, 2), 6.82 (t, J=9.2 Hz, 1), 3.90 (br s, 4), 3.68 (br s, 2), 3.16 (q, J=7.4 Hz, 2), 2.75 (br s, 4), 1.41 (t, J=7.35 Hz, 3). Also noted 5.1 (s), 3.4 (s), 2.2 (s), 1.6 (br s, H₂O), 1.3 (s).

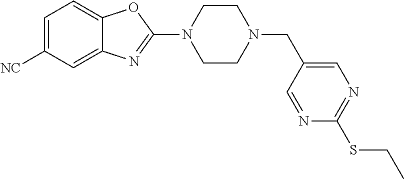 [SMILES: CCSc1ncc(CN2CCN(c3nc4cc(C#N)ccc4o3)CC2)cn1]

2-(4-((2-(ethylthio)pyrimidin-5-yl)methyl)piperazin-1-yl)benzo[d]oxazole-5-carbonitrile (JSF-4522): ¹H NMR (500 MHz, CDCl₃) δ 8.56 (s, 2), 7.59 (s, 1), 7.35 (dd, J=8.3, 14.3 Hz, 2), 3.85 (br s, 4), 3.64 (br s, 2), 3.19 (q, J=7.3 Hz, 2), 2.72 (br s, 4), 1.41 (t, J=7.3 Hz, 3). Also noted 1.2 (br s)

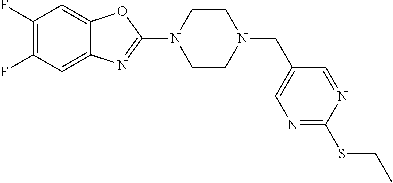 [SMILES: CCSc1ncc(CN2CCN(c3nc4cc(F)c(F)cc4o3)CC2)cn1]

2-(4-((2-(ethylthio)pyrimidin-5-yl)methyl)piperazin-1-yl)-5,6-difluorobenzo[d]oxazole (JSF-4525): ¹H NMR (500 MHz, CDCl₃) δ 8.59 (br s, 2), 7.16 (q, J=7.5 Hz, 2), 3.74 (br s, 6), 3.17 (q, J=7.3 Hz, 2), 2.64 (br s, 4), 1.41 (t, J=7.3 Hz, 3). Also noted 5.3 (s, DCM) 1.6 (br s, H₂O), 1.2 (s).

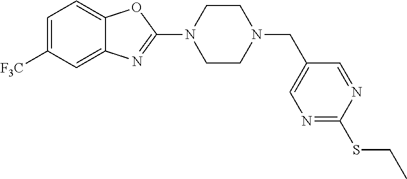 [SMILES: CCSc1ncc(CN2CCN(c3nc4cc(C(F)(F)F)ccc4o3)CC2)cn1]

2-(4-((2-(ethylthio)pyrimidin-5-yl)methyl)piperazin-1-yl)-5-(trifluoromethyl)benzo[d]oxazole (JSF-4526): ¹H NMR (500 MHz, CDCl₃) δ 8.51 (br s, 2), 7.58 (s, 1), 7.32 (s, 2), 3.76 (br s, 4), 3.52 (br s, 2), 3.17 (q, J=7.3 Hz, 2), 2.61 (br s, 4), 1.41 (t, J=7.3 Hz, 3). Also noted, 1.6 (br s, H₂O). ¹⁹F NMR (470 MHz, CDCl₃) δ −107.71 (s).

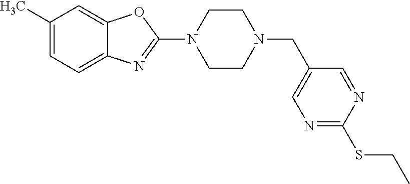 [SMILES: CCSc1ncc(CN2CCN(c3nc4ccc(C)cc4o3)CC2)cn1]

2-(4-((2-(ethylthio)pyrimidin-5-yl)methyl)piperazin-1-yl)-6-methylbenzo[d]oxazole (JSF-4527): ¹H NMR (500 MHz, CDCl₃) δ 8.54 (br s, 2), 7.25 (s, 1), 7.09 (s, 1), 7.00 (d, J=7.8 Hz, 1) 3.76 (br s, 6), 3.17 (q, J=7.3 Hz, 2), 2.63 (br s, 4), 2.41 (s, 3), 1.41 (t, J=7.3 Hz, 3). Also noted, 1.6 (br s, H2O). C19H23N5OS [M+H]+=370.1702; Observed [M+H]+=370.1697.

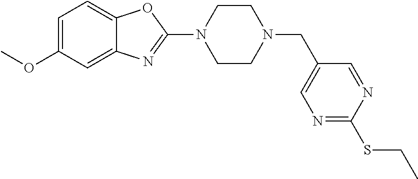 [SMILES: CCSc1ncc(CN2CCN(c3nc4cc(OC)ccc4o3)CC2)cn1]

2-(4-((2-(ethylthio)pyrimidin-5-yl)methyl)piperazin-1-yl)-5-methoxybenzo[d]oxazole (JSF-4528): ¹H NMR (500 MHz, CDCl₃) δ 8.53 (br s, 2), 7.15 (d, J=8.8 Hz, 1), 6.94-6.96 (m, 1), 3.81 (m, 7), 3.51 (br s, 2), 3.17 (q, J=7.3 Hz, 2), 2.59 (br s, 4), 2.41 (s, 3), 1.41 (t, J=7.3 Hz, 3). Also noted 1.6 (br s, H₂O), 1.2 (s). C19H23N5O2S [M+Na]+=408.1462; Observed [M+Na]+=408.1468.

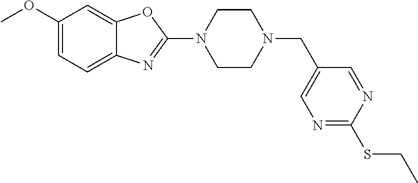 [SMILES: CCSc1ncc(CN2CCN(c3nc4ccc(OC)cc4o3)CC2)cn1]

2-(4-((2-(ethylthio)pyrimidin-5-yl)methyl)piperazin-1-yl)-6-methoxybenzo[d]oxazole (JSF-4534): ¹H NMR (500 MHz, CDCl₃) δ 8.62 (s, 2), 7.27 (s, 1), 6.89 (d, J=2.2 Hz, 1), 6.79 (d, J=8.5 Hz, 1), 3.92 (br s, 2), 3.81 (s, 3), 3.70 (br s, 4), 3.17 (q, J=7.4 Hz, 2), 2.64 (br s, 4), 1.41 (t, J=7.4 Hz, 3). Also noted 1.6 (br s, H₂O), 1.3 (t), 1.2 (s). C19H23N5O2S [M+H]+=386.1672; Observed [M+H]+=386.1645.

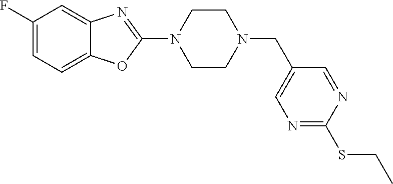 [SMILES: CCSc1ncc(CN2CCN(c3nc4cc(F)ccc4o3)CC2)cn1]

2-(4-((2-(ethylthio)pyrimidin-5-yl)methyl)piperazin-1-yl)-5-fluorobenzo[d]oxazole (JSF-4535): ¹H NMR (500 MHz, DMSO-d₆) δ 8.53 (s, 2), 7.38 (q, J=4.5 Hz, 1), 7.11 (dd, J=9.2, 2.5 Hz, 1), 6.81 (td, J=9.9, 2.6 Hz, 1), 3.60 (m, 4), 3.52 (s, 2), 3.11 (q, J=7.3 Hz, 2), 2.52 (s, 4), 1.32 (t, J=7.3 Hz, 3). Also noted 10.0 (s), 9.0 (s), 3.3 (s, H₂O), and 3.2 (q). C₁₈H₂₀FN₅OS [M+H]+=374.1472; Observed [M+H]+=374.1445.

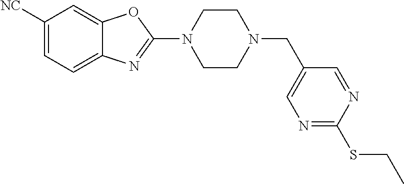 [SMILES: CCSc1ncc(CN2CCN(c3nc4ccc(C#N)cc4o3)CC2)cn1]

2-(4-((2-(ethylthio)pyrimidin-5-yl)methyl)piperazin-1-yl)benzo[d]oxazole-6-carbonitrile (JSF-4538): ¹H NMR (500 MHz, DMSO-d₆) δ 8.58 (s, 2), 7.94 (s, 1), 7.60 (dd, J=8.1, 2.0 Hz, 1), 7.39 (d, J=8.2 Hz, 1), 3.65-3.67 (m, 4), 3.53 (s, 2), 3.11 (q, J=7.3 Hz, 2), 2.51-2.53 (m, 4), 1.31 (t, J=7.3 Hz, 3). Also noted 3.3 (s, H₂O). C₁₉H₂₀N₆OS [M+H]+=381.1519; Observed [M+H]+=381.1493.

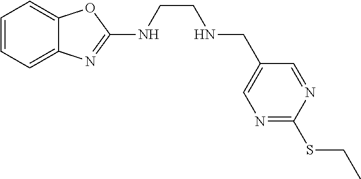 [SMILES: CCSc1ncc(CNCCNc2nc3ccccc3o2)cn1]

N¹-(benzo[d]oxazol-2-yl)-N²-((2-(ethylthio)pyrimidin-5-yl)methyl)ethane-1,2-diamine (JSF=4547): ¹H NMR (500 MHz, CD₃CN) δ 8.64 (s, 2), 7.41 (d, J=8.0 Hz, 1), 7.34 (d, J=7.7 Hz, 1), 7.28 (t, J=7.6 Hz, 1), 7.19 (t, J=7.9 Hz, 1), 4.19 (s, 2), 3.80 (t, J=4.7 Hz, 2), 3.39 (t, J=5.0 Hz, 2), 3.13 (q, J=7.3 Hz, 2), 1.35 (t, J=7.3 Hz, 3), Two protons unaccounted for. Also noted 5.1 (br s), 4.7 (s), 4.6 (s). C₁₆H₁₉N₅OS [M+H]+=330.1410; Observed [M+H]+=330.1393.

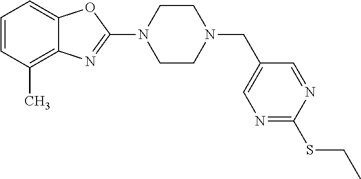 [SMILES: CCSc1ncc(CN2CCN(c3nc4c(C)cccc4o3)CC2)cn1]

2-(4-((2-(ethylthio)pyrimidin-5-yl)methyl)piperazin-1-yl)-4-methylbenzo[d]oxazole (JSF-4551): ¹H NMR (500 MHz, acetone-d₆) δ 8.58 (s, 2), 7.13 (d, J=7.8 Hz, 1), 6.97 (d, J=7.6 Hz, 1), 6.91 (t, J=7.8 Hz, 1), 3.70 (t, J=4.8 Hz, 4), 3.60 (s, 2), 3.15 (q, J=7.3 Hz, 2), 2.62 (t, J=4.8 Hz, 4), 2.40 (s, 3), 1.36 (t, J=7.3 Hz, 3). Also noted 5.1 (s, DCM), 3.3 (s), 2.8 (br s, H₂O), 1.30 (s), 0.9 (br s). C19H23N5OS [M+H]+=370.1723; Observed [M+H]+=370.1696.

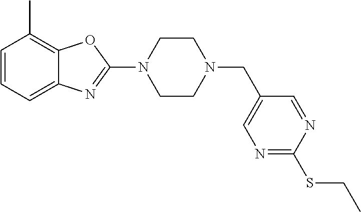 [SMILES: CCSc1ncc(CN2CCN(c3nc4cccc(C)c4o3)CC2)cn1]

2-(4-((2-(ethylthio)pyrimidin-5-yl)methyl)piperazin-1-yl)-7-methylbenzo[d]oxazole (JSF-4562): ¹H NMR (500 MHz, CDCl3) δ 8.54 (br s, 2), 7.20 (d, J=7.7 Hz, 1), 7.08 (t, J=7.6 Hz, 1), 6.86 (d, J=7.2 Hz, 1), 3.79 (br s, 4), 3.55 (br s, 2), 3.17 (q, J=7.4 Hz, 2), 2.59 (br s, 4), 2.42 (s, 3), 1.40 (t, J=7.3 Hz, 3). Also noted 5.2 (s), 3.3 (s), 1.6 (br s, H₂O), 1.2 (s), 0.8 (m).

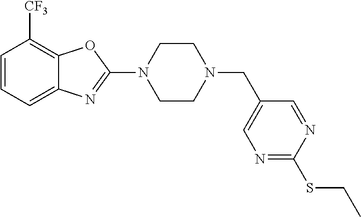 [SMILES: CCSc1ncc(CN2CCN(c3nc4cccc(C(F)(F)F)c4o3)CC2)cn1]

2-(4-((2-(ethylthio)pyrimidin-5-yl)methyl)piperazin-1-yl)-7-(trifluoromethyl)benzo[d]oxazole (JSF-4601): H NMR (500 MHz, d₆-Acetone) δ 8.58 (s, 2), 7.51 (d, J=7.6 Hz, 1), 7.33 (t, J=7.8 Hz, 1), 7.28 (d, J=7.7 Hz, 1), 3.78-3.74 (m, 4), 3.61 (s, 2), 3.17-3.12 (m, 2), 2.67-2.63 (m, 4), 1.36 (t, J=7.3 Hz, 3). Also noted 3.3 (s), 2.8 (br s, H₂O), 1.9 (s), 1.3 (s). C₁₉H₂₀F₃N₅OS [M+H]+=424.1441; Observed [M+H]+=424.1414.

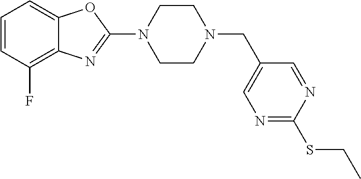 [SMILES: CCSc1ncc(CN2CCN(c3nc4c(F)cccc4o3)CC2)cn1]

2-(4-((2-(ethylthio)pyrimidin-5-yl)methyl)piperazin-1-yl)-4-fluorobenzo[d]oxazole (JSF-4602): ¹H NMR (500 MHz, d₆-acetone) δ 8.84 (br s, 2), 7.22 (d, J=7.9 Hz, 1), 7.09-7.03 (m, 1), 6.99 (t, J=9.3 Hz, 1), 4.00 (br s, 8), 3.15 (d, J=7.3 Hz, 2), 2.79 (d, J=15.3 Hz, 2), 1.36 (t, J=7.3 Hz, 3). Also noted 5.2 (s), 5.1 (s), 3.3 (s), 1.3 (s), 0.9 (s). C18H20FN5OS [M+H]+=374.1473; Observed [M+H]+=374.1443.

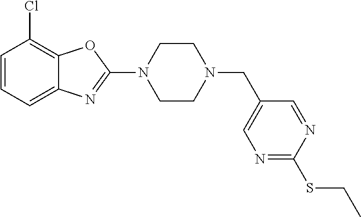 [SMILES: CCSc1ncc(CN2CCN(c3nc4cccc(Cl)c4o3)CC2)cn1]

7-chloro-2-(4-((2-(ethylthio)pyrimidin-5-yl)methyl)piperazin-1-yl)benzo[d]oxazole (JSF-4603): ¹H NMR (500 MHz, d₆-acetone) δ 8.60 (s, 2), 7.20 (d, J=7.7 Hz, 1), 7.15 (t, J=7.9 Hz, 1), 7.04 (d, J=7.5 Hz, 1), 3.76 (br s, 4), 3.63 (br s, 2), 3.15 (q, J=7.3 Hz, 2), 2.67 (br s, 4), 1.36 (t, J=7.3 Hz, 3). Also noted 5.1 (s, DCM), 3.3 (s), 2.8 (br s, H₂O), 1.2 (br s). C18H20ClN5OS [M+H]+=390.1155; Observed [M+H]+=390.1153.

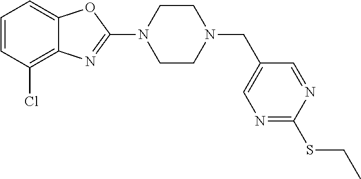 [SMILES: CCSc1ncc(CN2CCN(c3nc4c(Cl)cccc4o3)CC2)cn1]

4-chloro-2-(4-((2-(ethylthio)pyrimidin-5-yl)methyl)piperazin-1-yl)benzo[d]oxazole (JSF-4604): ¹H NMR (500 MHz, CD₃CN) δ 8.63 (s, 2), 7.30 (d, J=8.1 Hz, 1), 7.22 (d, J=7.8 Hz, 1), 7.05 (t, J=8.1 Hz, 1), 4.19 (s, 2), 3.95 (br s, 4), 3.30 (br s, 4), 3.15 (q, J=7.3 Hz, 2), 1.36 (t, J=7.3 Hz, 3). C18H20ClN5OS [M+H]+=390.1177; Observed [M+H]+=390.1153.

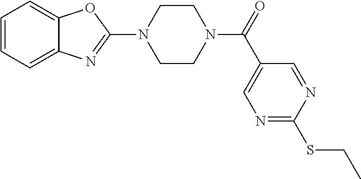 [SMILES: CCSc1ncc(C(=O)N2CCN(c3nc4ccccc4o3)CC2)cn1]

(4-(benzo[d]oxazol-2-yl)piperazin-1-yl)(2-(ethylthio)pyrimidin-5-yl)methanone (JSF-4683): ¹H NMR (500 MHz, CDCl₃) δ 8.62 (s, 2), 7.53 (dd, J=15.7, 7.9 Hz, 1), 7.37 (d, J=8.1 Hz, 1), 7.31 (dd, J=10.5, 4.2 Hz, 1), 7.25-7.19 (m, 1), 4.04 (br s, 4), 3.85 (d, J=22.7 Hz, 4), 3.19 (q, J=7.4 Hz, 2), 1.42 (t, J=7.4 Hz, 3). Also noted 9.0 (s), 1.3 (t). HRMS (ESI) calc'd for C₁₈H₁₉N₅O₂S+H+=370.1339 found 370.1338.

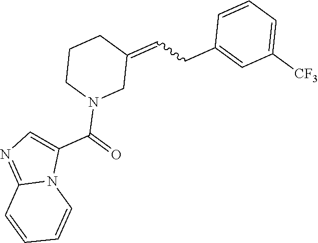 [SMILES: O=C(c1cnc2ccccn12)N1CCCC(=CCc2cccc(C(F)(F)F)c2)C1]

Imidazo[1,2-a]pyridin-3-yl(3-(2-(3-(trifluoromethyl)phenyl)ethylidene)piperidin-1-yl)methanone (JSF-4714): ¹H NMR (500 MHz, CDCl₃) δ 8.98 (d, J=7.0 Hz, 1), 7.89 (s, 1), 7.71 (m, 1), 7.41 (dd, J=15.8, 7.7 Hz, 3), 7.35-7.28 (m, 2), 6.97 (m, 1), 5.46 (t, J=7.5 Hz, 1), 4.47 (s, 2), 3.93 (m, 2), 3.51 (d, J=7.0 Hz, 2), 2.43 (t, J=6.1 Hz, 2), 1.80 (m, 2), Also noted δ 1.2 (br s), 0.9 (m). HRMS Calculated for C₂₂H₂₁F₃N₃O [M+H]⁺=400.1637, observed 400.1632.

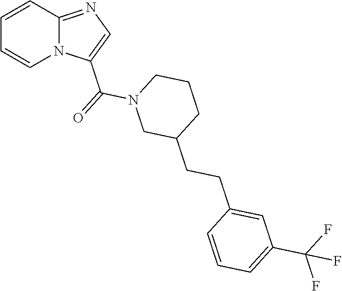 [SMILES: O=C(c1cnc2ccccn12)N1CCCC(CCc2cccc(C(F)(F)F)c2)C1]

Imidazo[1,2-a]pyridin-3-yl(3-(3-(trifluoromethyl)phenethyl)piperidin-1-yl)methanone (JSF-4706/C5): ¹H NMR (500 MHz, CDCl₃) δ 9.06 (d, J=6.9 Hz, 1), 8.07 (d, J=8.8 Hz, 1), 7.98 (s, 1), 7.68 (t, J=7.7 Hz, 1), 7.44 (d, J=8.5 Hz, 2), 7.37 (dd, J=12.9, 7.7 Hz, 2), 7.23 (t, J=7.0 Hz, 1), 4.47-4.33 (m, 2), 3.18 (s, 1), 2.87 (d, J=10.5 Hz, 1), 2.74 (d, J=8.8 Hz, 2), 2.04 (d, J=13.1 Hz, 1), 1.86 (d, J=16.4 Hz, 1), 1.68-1.58 (m, 3), 0.89-0.82 (m, 2), Also noted δ 1.25 (br s, grease). HRMS Calculated for C₂₂H₂₂F₃N₃O₃[M+H]⁺=402.1793, observed 402.1786.

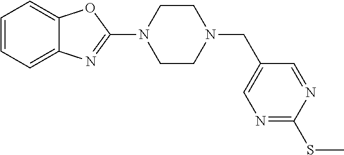 [SMILES: CSc1ncc(CN2CCN(c3nc4ccccc4o3)CC2)cn1]

2-(4-((2-(methylthio)pyrimidin-5-yl)methyl)piperazin-1-yl)benzo[d]oxazole (JSF-4730): ¹H NMR (500 MHz, CD₃CN) δ 8.51 (s, 2), 7.28 (dd, J=14.6, 7.9 Hz, 2), 7.15 (t, J=8.1 Hz, 1), 7.02 (t, J=8.3 Hz, 1), 3.63 (m, 4), 3.50 (s, 2), 2.55 (m, 4), 2.53 (s, 3). Also noted 4.0 (q, EtOAc), 2.1 (s, H₂O), and 1.2 (t, EtOAc). HRMS Calculated for C₁₇H₂₀N₅OS [M+H]⁺=342.1390, observed 342.1384.

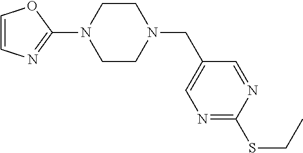 [SMILES: CCSc1ncc(CN2CCN(c3ncco3)CC2)cn1]

2-(4-((2-(ethylthio)pyrimidin-5-yl)methyl)piperazin-1-yl)oxazole (JSF-4747): ¹H NMR (500 MHz, CD₃CN) 8.47 (s, 2H), 7.31 (s, 1H), 6.78 (s, 1H), 3.46 (s, 2H), 3.41 (m, 4H), 3.12 (q, J=7.3 Hz, 2H), 2.49 (m, 4H), 1.34 (t, J=7.3 Hz, 3H). HRMS Calculated for C₁₄H₂₀N₅OS [M+H]⁺=306.1390, observed 306.1395.

### Results

Reporter-Based Chemical Screen Identifies Compounds that Modulate Mtb Response to Environmental Chloride Levels

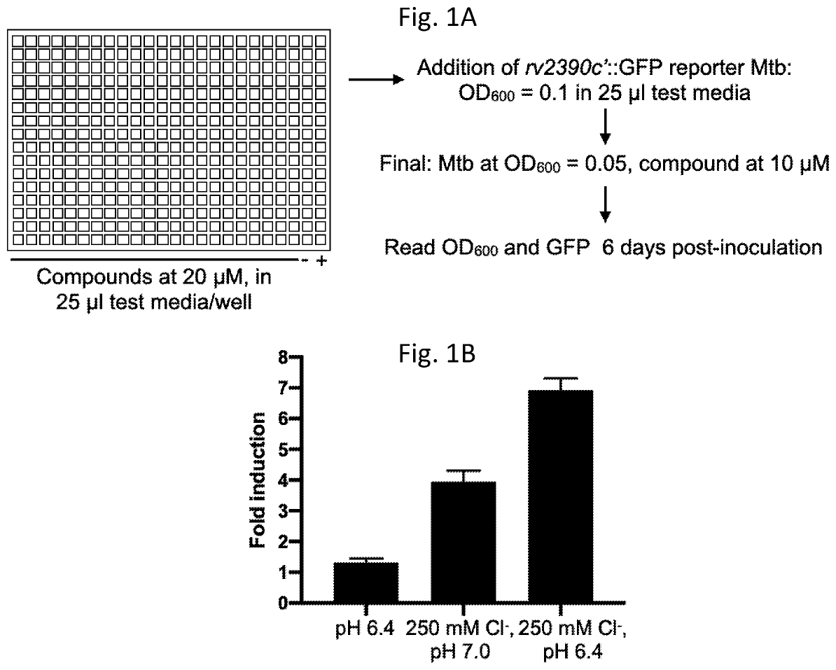

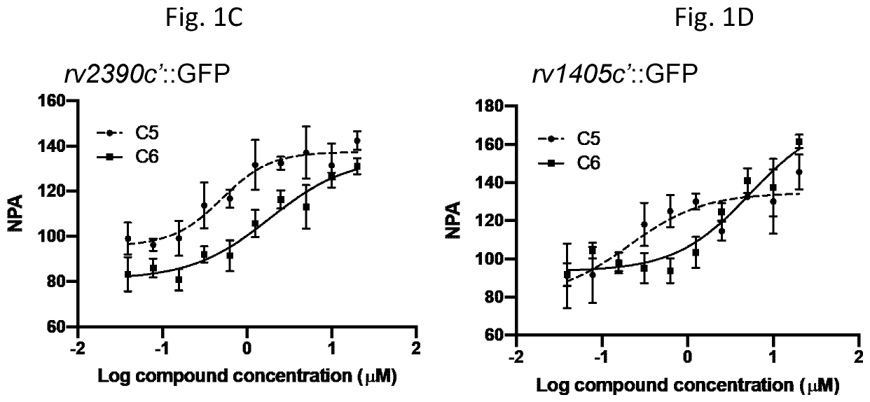

To identify compounds that modulate Mtb response to environmental chloride (Cl⁻) levels, we exploited our previously described Cl⁻ and pH-responsive rv2390c′::GFP reporter Mtb strain in a 384-well format fluorescence-based screen (FIG. 1A). GFP signal in this reporter strain is induced upon bacterial exposure to high Cl⁻ concentrations ([Cl⁻]) or acidic pH, with a synergistic response observed in the presence of both signals (Tan et al, 2013). The test condition of 250 mM NaCl, pH 6.4 was chosen to maximize reporter dynamic range while ensuring compound hits reflected effects on Mtb Cl⁻ response (FIG. 1B) (Tan et al, 2013). A total of 50,816 compounds from The Rockefeller University's High Throughput and Spectroscopy Center were screened, with a robust average Z′-factor of 0.71±0.05. The threshold for calling hits was set at 3 standard deviations from the mean, and resulted in 69 hits that increased reporter induction and 67 hits that decreased reporter induction as compared to DMSO-treated control samples. As inhibition of Mtb growth confounds analysis of rv2390c′::GFP reporter induction, 43/67 “hits” that decreased reporter signal upon exposure to high [Cl⁻] but also exhibited significant inhibition of bacterial growth were excluded from further follow-up.

As a first validation of the results from the primary screen, the rv2390c′::GFP assay was repeated with a dose response curve for all called hits in triplicate. In accord with the robustness of the screen, the majority of hits were validated, with 68/69 compounds that increased reporter induction and 21/24 compounds that decreased reporter induction exhibiting the phenotype observed in the primary screen (FIG. 1C). These validated compounds were then used in assays with a second, independent, Cl⁻/pH-responsive reporter, rv1405c′::GFP, to focus on compounds that more generally affect Mtb Cl⁻ response versus those specific for effects on rv2390c expression. In this secondary screen, 49/68 compounds that increased rv2390c′::GFP reporter induction upon exposure to high [Cl⁻], and 18/21 compounds that decreased rv2390c′::GFP reporter induction, also showed similar phenotypes with the rv1405c′::GFP reporter (FIG. 1D).

Liquid chromatography-mass spectrometry (LC-MS) carried out on 32 resupplied hit compounds (16 that increased reporter induction and 16 that decreased reporter induction) was utilized to validate compound identity and purity. In all cases, the observed parent mass was consistent with the hit chemical structure. The chromatograms demonstrated 13/16 compounds in each class showed >99% purity, and no compound exhibited less than 88% purity.

Compounds Identified that Inhibit Mtb Growth in Host Macrophages

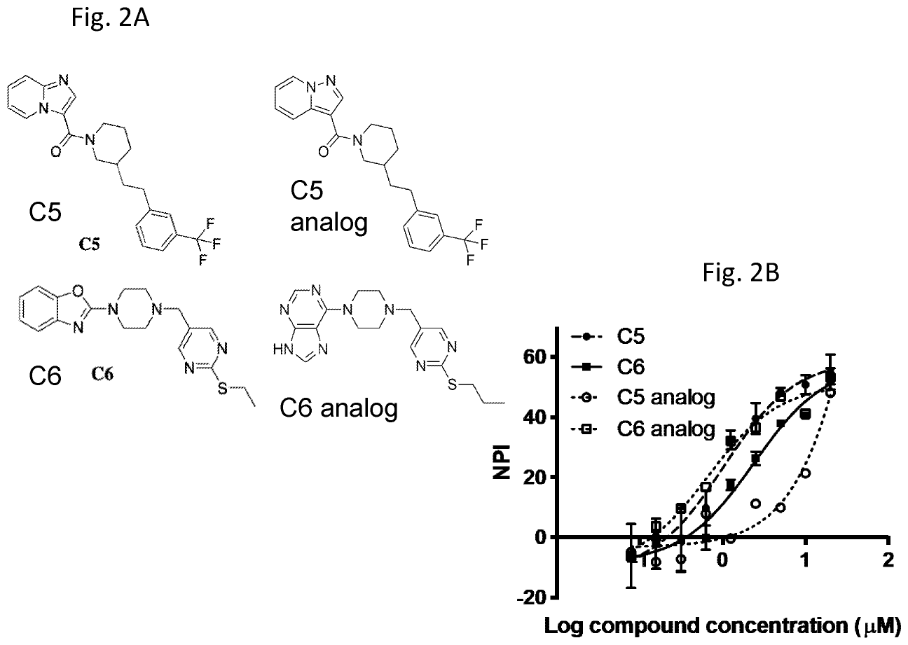

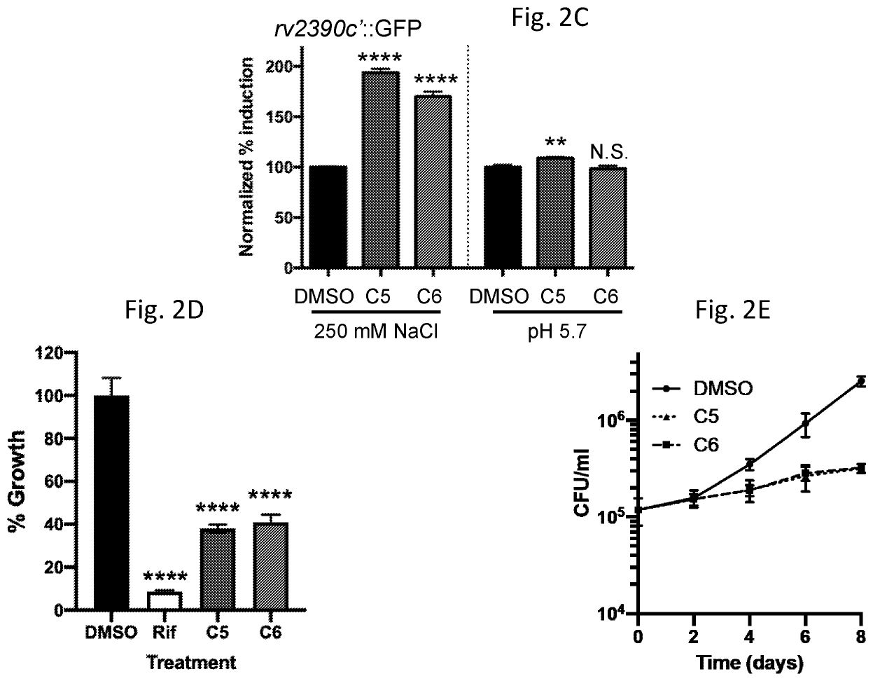

With these hit compounds identified, we next conducted a tertiary screen to examine the effects of the selected compounds on the ability of Mtb to grow in host macrophages, and to aid in prioritization of compounds for follow-up study. J774 murine macrophage-like cells were seeded in 384-well plates, infected with Mtb constitutively expressing the fluorophore monomeric Kusabira Orange (mKO), and treated with compounds in a dose-response curve. Mtb colonization of the host cells was tracked by analyzing mKO fluorescence six days post-infection, and compared to DMSO-treated samples or cells treated with 5 μM rifampicin, a first-line Mtb drug. With the 5 μM rifampicin samples set as 100% inhibition of Mtb growth, 11 compounds were found to inhibit Mtb growth in J774 cells >20 at 20 M (Table 1). Among these 11 compounds, there were two pairs of compounds in which the paired structures were closely related analogs (FIG. 2A), and where the phenotypes observed closely matched between the paired compounds. In each case, rv2390c′::GFP reporter signal was increased upon Mtb exposure to high [Cl⁻] and in the presence of compound treatment, and bacterial growth in J774 host cells was inhibited 50% at 20 μM compound (FIG. 2B). Based on the strength of these phenotypes, one compound from each of these pairs (termed “C5” and “C6”) was selected for continued follow-up.

TABLE 1
Inhibition of MtB growth in J774 cells.

| | | % inhibition | % inhibition |
|---|---|---|---|
| | rv2390c′::GFP response | compared to Rif | compared to Rif |
| Structure | phenotype | in J774 at 20 μM | in J774 at 10 μM |
| (C5) | Increases induction in high | 55 ± 5.8 | 50.9 ± 3.2 |
| | chloride | | |
| (C5 analog) | Increases induction in high | 48.2 ± 1.5 | 21.4 ± 7.7 |
| | chloride | | |
| (C6) | Increases induction in high | 53.6 ± 2.7 | 40.5 ± 0.6 |
| | chloride | | |
| (C6 analog) | Increases induction in high | 53.2 ± 1.6 | 41.2 ± 0.6 |
| | chloride | | |
| | Increases induction in high | 25.3 ± 4.1 | 22.2 ± 0.2 |
| | chloride | | |
| | Increases induction in high | 34.9 ± 5.2 | 21.4 ± 7.4 |
| | chloride | | |
| | Increases induction in high | 36.1 ± 0.1 | 4.8 ± 0.1 |
| | chloride | | |
| | Increases induction in high | 30.4 ± 2.4 | No apparent |
| | chloride (did not increase | | inhibition |
| | induction of rv1405c′::GFP | | |
| | reporter to high chloride in | | |
| | secondary screen) | | |
| | Decreases induction in high | 61.5 ± 2.6 | 23.9 ± 12.9 |
| | chloride | | |
| | Decreases induction in high | 28.8 ± 4.5 | 12.1 ± 10.5 |
| | chloride (did not appear to have | | |
| | robust effect on induction of | | |
| | rv1405c′::GFP reporter to high | | |
| | chloride in secondary screen) | | |
| | Decreases induction in high | 20.9 ± 6.9 | 9.6 ± 0.7 |
| | chloride | | |

Commercially purchased C5 and C6 reproduced the increased rv2390c′::GFP reporter signal phenotype upon Mtb exposure to high [Cl⁻], as well as the phenotype of Mtb growth inhibition during J774 infection (FIGS. 2C and 2D). Further, treatment of primary murine bone marrow-derived macrophages (BMDM) with 10 μM C5 or C6 resulted in a significant decrease in bacterial load observed versus DMSO mock-treated samples in an eight-day infection time-course (FIG. 2E).

Together, these results identified C5 and C6 as compounds that perturb Mtb response to environmental Cl⁻, and that inhibit Mtb growth in host macrophages.

### Compound C6 Exhibits Promising Drug-Like Properties

Given the marked ability of C5 and C6 to inhibit Mtb growth in host macrophages, we pursued examination of their physiochemical and pharmacokinetic properties with a long-term goal of assessing their potential for in vivo study of efficacy. Both compounds exhibited acceptable (Inoyama et al, 2018) kinetic aqueous solubility (S) in pH 7.4 phosphate buffered saline (PBS) with values of 6.67 μM (C5) and 76.4 μM (C6). Stability, as judged by half-life (t_{1/2}) in the presence of mouse liver microsomes was assessed for each compound to be below the target value of 60 min (Inoyama et al, 2018). The C6 t_{1/2} value of 48.5 min was superior to that for C5 (t_{1/2}=4.98 min). The greater solubility and metabolic stability of C6 as compared to C5 were reflected in its higher plasma exposure observed in CD-1 mice (n=2) post a single 5 mg/kg oral (po) dose. Specifically, C6 exhibited greater area under the curve over 5 h (AUC_{0-5 h}=5481 h*ng/ml) than C5 (AUC_{0-5 h}=42 h*ng/ml).

With the superior pharmacokinetic (PK) profile at 5 mg/kg, C6 was further profiled, beginning with the determination of its plasma AUC_{0-5 h} to be 25713 h*ng/ml with a single 25 mg/kg po dose. Its five-hour partitioning between plasma and lung was characterized by a C_{lung}/Cₚₗₐₛₘₐ=0.88. C6 was shown to lack significant toxicity (CC₅₀>50 μg/ml; where CC₅₀=minimum compound concentration to inhibit cell growth by 50%) to in vitro cultured Vero cells, a widely utilized model mammalian cell line for compound toxicity assays (Perryman et al, 2018). C6 exhibited a slightly shorter t_{1/2} (37.6 min) in the presence of human liver microsomes as compared to mouse liver microsomes. The compound was characterized by high, but not prohibitively large, protein binding to mouse (97.0%) or human (99.2%) plasma. C6 exhibited sufficient stability in the presence of mouse (>99.9% remaining) or human (94.6% remaining) plasma after 5 h incubation. Inhibition of human CYP 450 enzymes was negligible, with 10 μM C6 affording <1, 25.7, 30.3, 10.5, and <1% inhibition of the 1A2, 2C9, 2C19, 2D6, and 3A4/5 isoforms respectively. Finally, hERG inhibition was quantified by an IC₅₀=1.2±0.1 μM. Reduction of hERG inhibition, along with enhancements in mouse liver microsome stability and mouse oral exposure, will thus be goals in future analogs for this chemical series.

Drug-like properties for several compounds are compared in Table 2 for mice subjected to a single 25 mg/kg po dose of the listed compound formulated in 5% DMA/60% PEG3000/35% D5W.

TABLE 2
C6 analog structure activity relationships for in vitro and in vivo properties
(TBD = to be determined).

| | | % Reduction of | | | | |
|---|---|---|---|---|---|---|
| | | Intracellular | | | | Kinetic |
| | | Infection | Mouse PK | Mouse PK | MLM | Aqueous |
| | | (J774.1 cells) | AUC₀₋₅ₕ | C_{lung}/Cₚₗₐₛₘₐ | t_{1/2} | Solubility S |
| Cmpd | R | at 10 μM cmpd | (h*ng/mL) | at t = 5 h | (min) | (mM) |
| C6 | H | 54.2 ± 2.6 | 25713 | 0.88 | 48.5 | 76.4 |
| JSF-4467 | 6-Cl | 50.6 ± 2.0 | 41675 | 0.36 | 54.3 | 8.3 |
| JSF-4525 | 5,6-diF | 46.3 ± 2.9^{b} | 29700 | 0.33 | 39.8 | 0.952 |
| JSF-4526 | 5-CF₃ | 52.4 ± 1.4 | 27546 | 0.44 | 32.0 | 38.6 |
| JSF-4527 | 6-CH₃ | 53.8 ± 2.1 | 16269 | 0.55 | 38.7 | 13.7 |
| JSF-4538 | 6-CN | 36.4 ± 2.6 | 98712 | 0.30 | 25.5 | TBD |

Structure-Activity Relationship Analysis Reveals that the Chloride Response Phenotype of C6 Tracks with Inhibition of Mtb Growth in Macrophages

To begin to probe some of the basic SAR for C6, we devised a scalable synthesis of C6 that could readily be extended to the synthesis of analogs. C6 was synthesized in three steps from commercially available 2-chlorobenzo[d]oxazole involving displacement with piperazine followed by reductive amination with commercial 2-(ethylthio)pyrimidine-5-carbaldehyde in 60.3% overall yield (FIG. 3A). This route was adapted through the use of 2-methoxypyrimidine-5-carbaldehyde, pyrimidine-5-carbaldehyde, or 4-(ethylthio)benzaldehyde to prepare JSF-4271, JSF-4297, and JSF-4300 respectively (FIG. 3B). 2-chlorobenzo[d]thiazole was substituted in the reaction scheme to afford JSF-4298 (FIG. 3B).

Tests of the C6 analogs in the rv2390c′::GFP reporter assay to determine compound modulation of Mtb response to Cl⁻ showed that analogs JSF-4271 and JSF-4297 had lost the ability demonstrated by the parental C6 compound to increase reporter GFP signal in the presence of high [Cl⁻] (FIG. 3C). Loss of this phenotype was also accompanied by loss of the ability to inhibit Mtb growth in J774 cells and primary murine bone marrow-derived macrophages (FIGS. 3D and 3E). While C6 and its analogs have no effect on Mtb growth in standard 7H9 media (FIG. 3F), we additionally examined the effect of the analogs on Mtb growth in cholesterol media, as C6 was also part of a previous independent screen for compounds that inhibited Mtb growth in cholesterol media (PubChem AID 1259343), and had been called as positive for growth inhibition in that screen (PubChem SID 340463129). We found that C6 indeed inhibited Mtb growth in cholesterol media (FIG. 3G). As with the rv2390c′::GFP reporter and growth in macrophage phenotypes, analogs JSF-4271 and JSF-4297 no longer inhibited Mtb growth in cholesterol media (FIG. 3G).

The impact of C6 on Mtb growth in cholesterol media but not standard 7H9 media, combined with its effect on induction of the rv2390c′::GFP reporter in high [Cl⁻] media, led us to analyze the activity of the rv2390c′::GFP reporter in untreated Mtb grown in cholesterol media. Cholesterol media alone had little effect on expression of the rv2390c′::GFP reporter (FIG. 2H), in accord with rv2390c not previously being reported to be part of the cholesterol regulon. Intriguingly however, we found that induction of the rv2390c′::GFP reporter in the presence of high [Cl⁻] was increased in the context of cholesterol media (FIG. 3H).

Together, these results reveal a potential intersection between Mtb response to Cl⁻ with its metabolism, and indicate the importance of the 2-ethylthio group, with analogs JSF-4298 and JSF-4300, which retain the 2-ethylthio group, both continuing to demonstrate the phenotypes seen with the parental C6 compound, in contrast to analogs JSF-4271 and JSF-4297.

C6 does not Alter Phagosome Maturation Characteristics

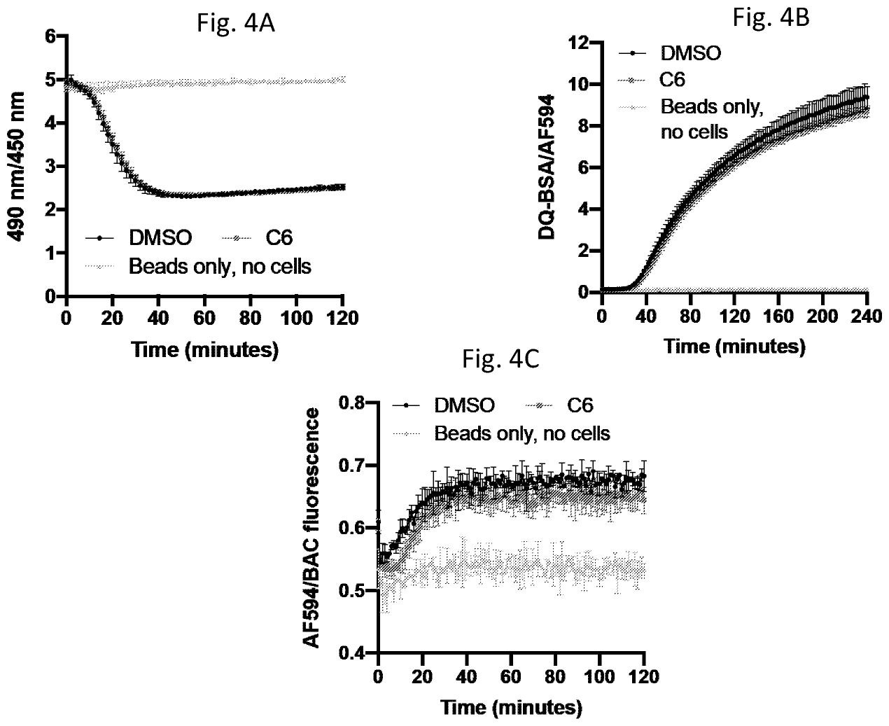

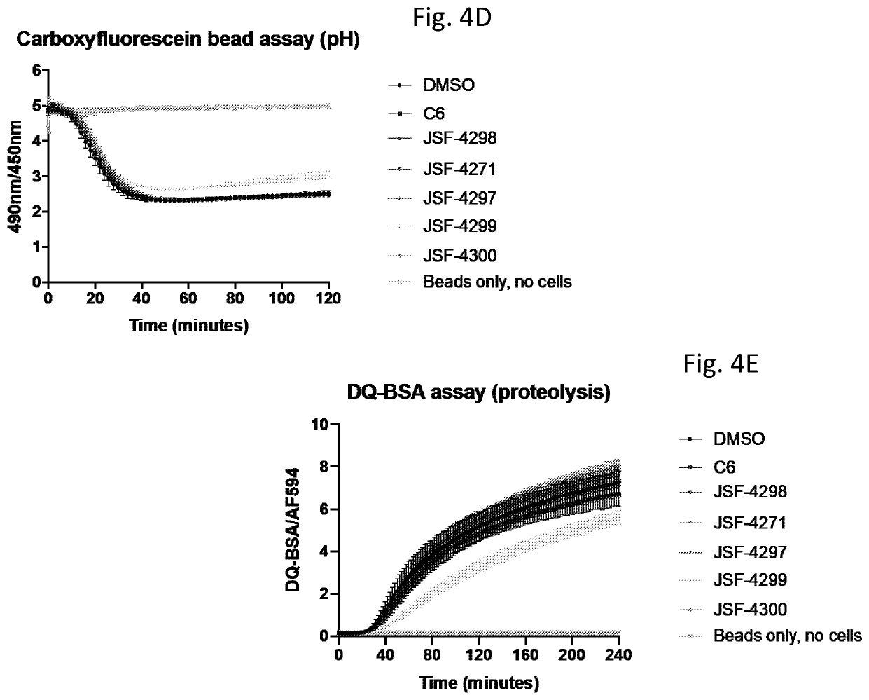

We next sought to determine if C6 exerted effects on the host cell, specifically on phagosome maturation characteristics, independent of infection. Carboxyfluorescein-linked silica beads enable ratiometric assessment of changes in macrophage phagosomal pH, via measurement of carboxyfluorescein fluorescence at pH-dependent versus pH-independent wavelengths (Buter et al, 2019; Dutta et al, 2020; Podinovskaia et al, 2013; Tan et al, 2017; Yates et al, 2008). As shown in FIG. 4A, addition of C6 did not alter macrophage phagosomal acidification. C6 also did not affect macrophage phagosomal proteolytic activity, as demonstrated using DQ-BSA/Alexa Fluor 594-linked silica beads (FIG. 4B) (Dutta et al, 2020; Podinovskaia et al, 2013; Yates et al, 2008). Finally, utilization of 10,10′-bis[3-carboxylpropyl]-9,9′-biacridinium (BAC)/Alexa Fluor 594-linked silica beads, reporting specifically on changes in Cl⁻ (Sonawane et al, 2002; Tan et al, 2013), demonstrated no significant effect of C6 on the increase in [Cl⁻] that occurs during macrophage phagosomal maturation (FIG. 4C). Together, these data demonstrate that C6 does not affect macrophage phagosomal maturation characteristics independent of infection.

FIG. 4D and FIG. 4E show a comparison of the phagosomal maturation characteristics for C6, JSF-4298, JSF-4271, JSF-4291, JSF-4299, and JSF-4300. These figures show the differential impact of JSF-4299 on phagosomal maturation properties, such as acidification and proteolysis. The data show that JSF-4299 phenocopies macrophage activation, where a delay in acidification and a decrease in the extent of proteolysis is similarly seen. Decreased proteolysis may promote antigen presentation, which may be beneficial for bacterial clearance, whereas the observed delay in acidification reflects a balance in the production of reactive oxygen species, which consume protons, and phagosomal acidification upon immune cell activation.

### C6 Accumulates in Mtb and Host Macrophages

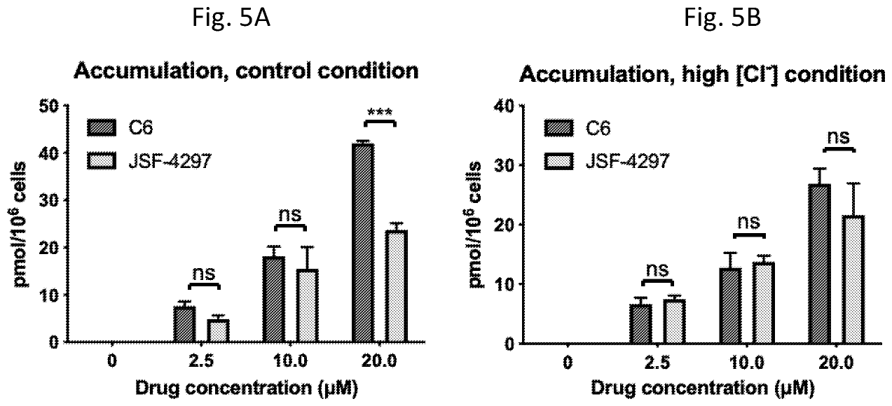

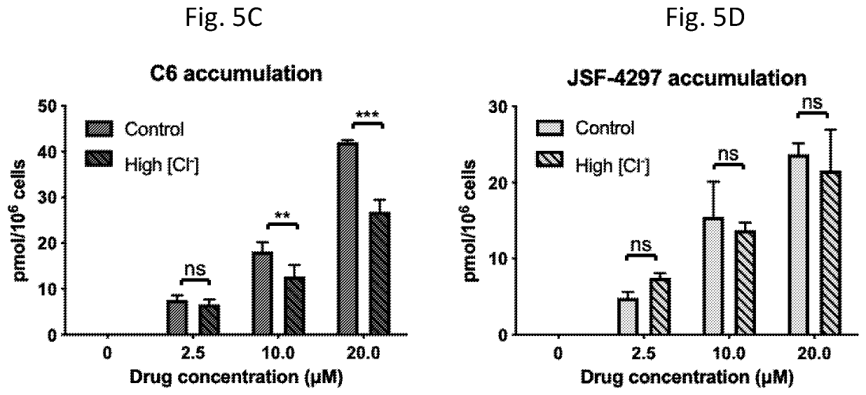

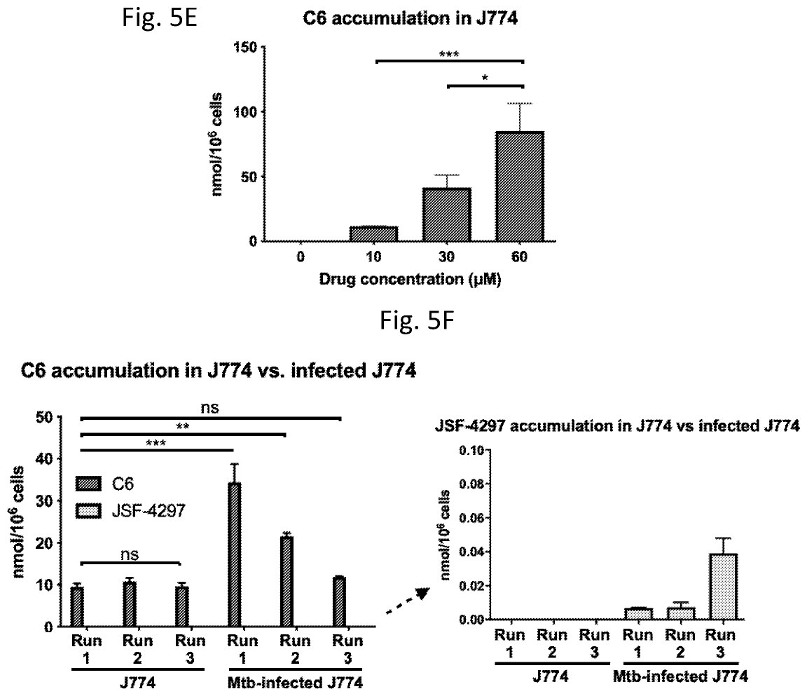

Since we observed the modulation of response to high [Cl⁻] in Mtb cells and inhibition of Mtb growth inside J774 macrophage-like cells by C6, we sought to confirm if these observed effects correlated with C6 accumulation in the Mtb and J774 cells. To quantify the intrabacterial accumulation of C6, we leveraged our recently developed label-free cell-based methodology that enables monitoring of drug accumulation/metabolism inside bacterial cells (Wang et al, 2020; Wang et al, 2019). Utilizing this intrabacterial drug metabolism (IBDM) platform, we measured C6 accumulation in Mtb after 24 h incubation of the bacteria under control or high [Cl⁻] media conditions, in the presence of a range of C6 concentrations (0-20 PM). This assay revealed dose-dependent C6 accumulation in the bacteria (FIGS. 5A-5C). Experiments with the inactive analog JSF-4297 showed that while this compound accumulated inside Mtb cells, it did so at levels lower than that observed with C6, and with no statistically significant difference in accumulation in the control versus high [Cl⁻] media conditions (FIGS. 5A, 5B, and 5D).

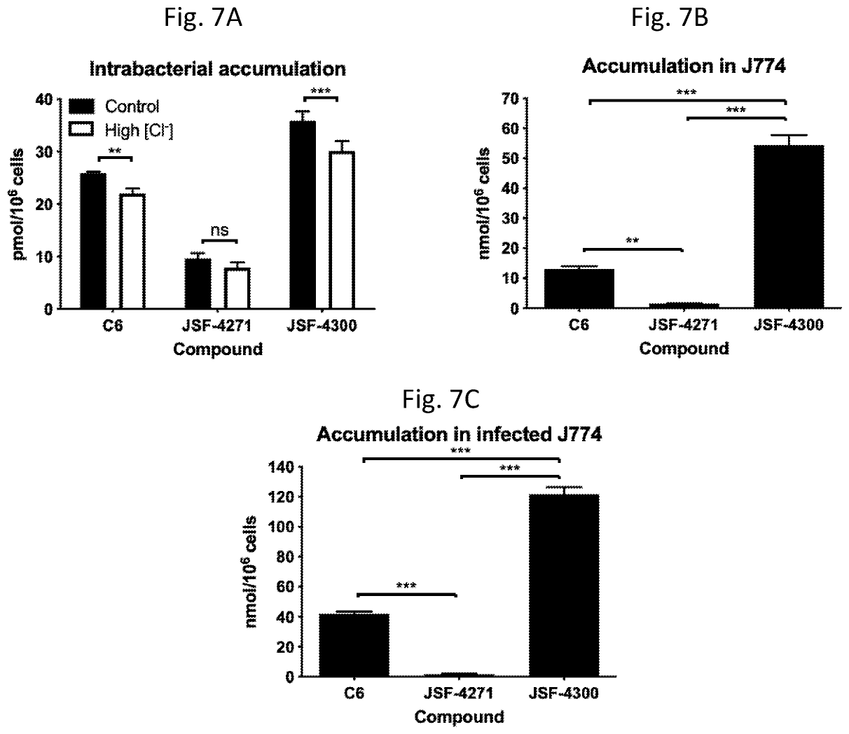

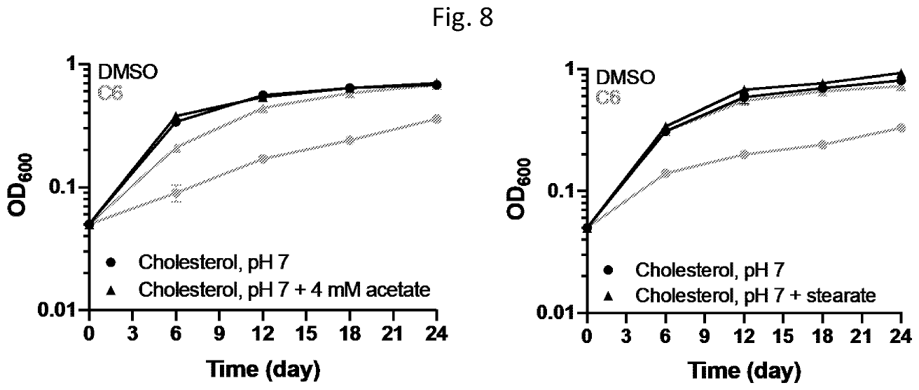

We then explored whether C6 could accumulate in the host J774 cells. With a modified protocol, we incubated J774 cells with 0-60 μM C6 for 24 h. A dose-dependent accumulation of C6 in J774 cells, similar to that observed in Mtb, was observed (FIG. 5E). We further confirmed C6 accumulation in the Mtb-infected J774 cells after incubating the cells with 10 μM C6 for 24 h (FIG. 5F), extending our scope to systems with bacteria-infected eukaryotic cells. There was a trend of greater C6 accumulation in the Mtb-infected J774 cells than uninfected J774 cells, although significant variation in accumulation was seen among experimental runs with the infected J774 cells (FIG. 5F). In striking contrast, we observed no detectable accumulation when we incubated J774 cells with JSF-4297 (FIG. 5F). In accord with these results, analyses of compound accumulation upon treatment with JSF-4271 (inactive analog) or JSF-4300 (active analog) showed decreased accumulation of JSF-4271 in Mtb, and minimal accumulation of JSF-4271 in host J774 cells or in samples extracted from Mtb-infected J774 cells, while robust accumulation was observed with the active analog JSF-4300 (FIG. 7). These results thus suggest an unexpected tie between compound structure and accumulation in bacterial and J774 host cells.

### C6 Inhibits Mtb Colonization In Vivo

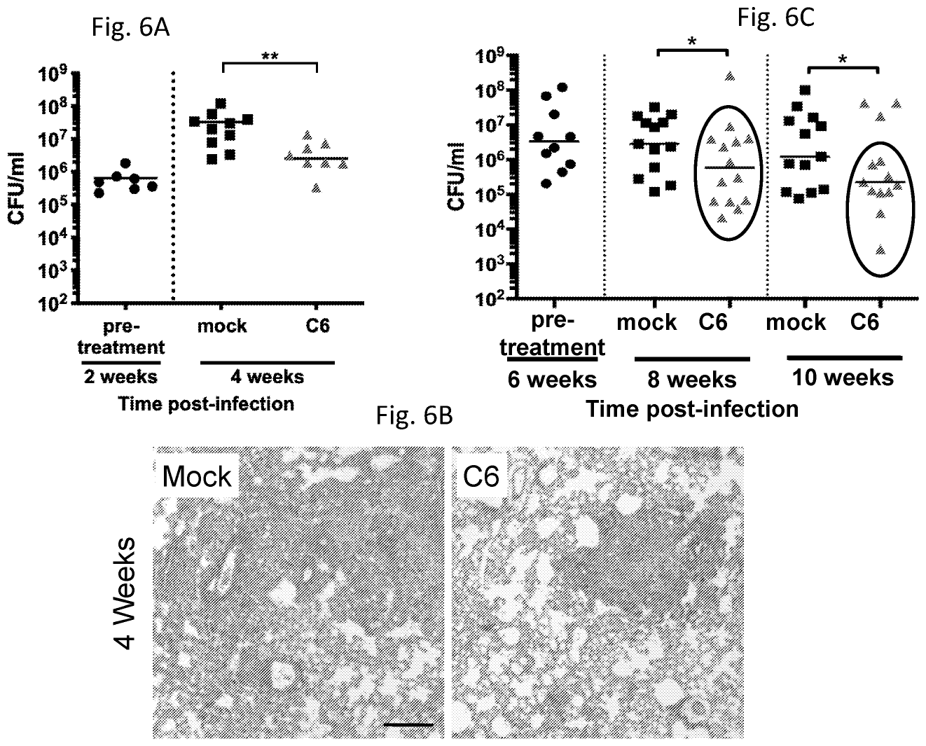

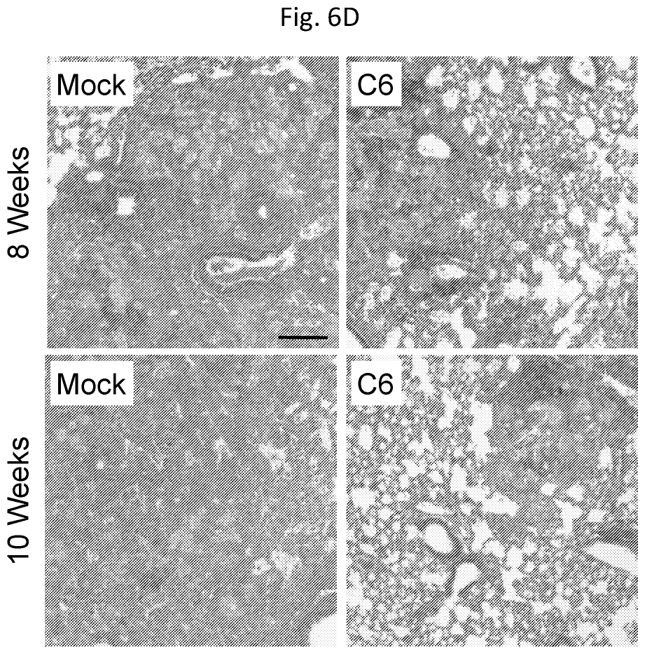

Given the promising results above of C6 inhibition of Mtb growth in host macrophages and its favorable PK profile, we finally sought to assess if C6 had in vivo efficacy. For these experiments, we utilized the C3HeB/FeJ murine infection model, as Mtb infection in these mice recapitulates key lesion types observed during human infection, and its use in studies of compound effects on Mtb treatment has thus become increasingly appreciated (Harper et al, 2012; Driver et al, 2012; Irwin et al, 2016; Lanoix et al, 2016a; Lanoix et al, 2016b; Lanoix et al, 2015). To begin, we tested the effects of C6 upon short-term Mtb infection, where treatment was initiated prior to the onset of adaptive immunity. Specifically, we infected C3HeB/FeJ mice with Mtb and allowed the infection to establish for two weeks. We then began treatment with 250 mg/kg C6 or vehicle (0.5% carboxymethyl cellulose+0.5% Tween 80) for a further two weeks, via oral gavage five times a week. Lungs were harvested two or four weeks post-infection and the bacterial load quantified. Excitingly, this early two-week treatment with C6 significantly inhibited Mtb growth in the mice lungs, with histological examination showing correspondingly less cellular infiltration (FIGS. 6A and 6B).

To extend on this result, we next pursued tests of the effect of C6 on a longer-term infection model in C3HeB/FeJ mice, to determine if inhibition of Mtb growth would still be observed post-formation of caseous necrotic lesions, a hallmark lesion type observed during human infection (Lanoix et al, 2015). This is of specific interest as the effects of antitubercular drugs have been shown to differ depending on whether caseous necrotic lesions are formed (Driver et al, 2012; Harper et al, 2012; Irwin et al, 2016; Lanoix et al, 2016b; Lanoix et al, 2015). For example, pyrazinamide and bedaquiline demonstrate uniform efficacy across animals in BALB/c mice, which do not form caseous necrotic lesions (Irwin et al, 2016; Lanoix et al, 2016b). In contrast, these two drugs demonstrate a heterogeneous response in C3HeB/FeJ mice, with a subset of mice failing to respond to treatment (Irwin et al, 2016; Lanoix et al, 2016b). In C3HeB/FeJ mice, caseous necrotic lesions can be observed 6 weeks post-infection with Mtb. We thus infected mice with Mtb and allowed the infection to establish for 6 weeks, before treating with 250 mg/kg C6 or vehicle for two or four weeks. Strikingly, C6 treatment resulted in a significant decrease in bacterial load at both the treatment time points, with corresponding improved histopathology (two or four weeks of treatment, eight or ten weeks of total infection time) (FIGS. 6C and 6D). Of note, a few outlier mice where bacterial load remained high were present within the C6-treated population (FIG. 6C). This is in accord with results previously observed with pyrazinamide and bedaquiline (Irwin et al, 2016; Lanoix, 2016b), and likely reflects the heterogeneity of the infection observed in C3HeB/FeJ mice. Further studies will be needed to elucidate the exact reasons for the failure of a subset of mice to respond to C6 treatment.

Together, these experiments demonstrate the ability of C6 to exert in vivo efficacy in a murine model that recapitulates key lesion types observed during human infection, significantly decreasing bacterial load and improving pathology.

### Comparative Phenotypic Response

Phenotypic response of the exemplary compounds are presented in Table 3.

TABLE 3
Comparison of phenotypic response.

| | | Inhibition of Mtb | Growth inhibition in |
|---|---|---|---|
| | rv2390c′::GFP Cl⁻ | growth in J774 cells | cholesterol media |
| Compound | response phenotype | phenotype | phenotype |
| JSF-4116 (C6) | Y | Y | Y |
| JSF-4298 | Y | Y− | Y |
| JSF-4271 | N | N | N |
| JSF-4297 | N | N | N |
| JSF-4299 | Y | Y | Y |
| JSF-4300 | Y | Y | Y |
| JSF-4467 | Y | Y | |
| JSF-4471 | Y | Y | |
| JSF-4477 | Y | Y+ | |
| JSF-4507 | Y | Y | |
| JSF-4509 | Y | Y+ | |
| JSF-4516 | N | Y | |
| JSF-4522 | | Y+ | |
| JSF-4525 | Y | Y | |
| JSF-4526 | Y | Y | |
| JSF-4527 | Y | Y | |
| JSF-4528 | N | Y+ | |
| JSF-4534 | | Y+ | |
| JSF-4538 | Y | Y+ | |
| JSF-4547 | N | N | |
| JSF-4551 | Y | Y | |
| JSF-4562 | Y | Y | |
| JSF-4601 | Y | Y− | |
| JSF-4602 | Y | Y | |
| JSF-4603 | Y | Y | |
| JSF-4604 | Y | Y | |
| JSF-4683 | N | | |
| JSF-4730 | Y | Y | |
| JSF-4747 | Y | | |

N = phenotype lost compared to C6
Y = phenotype present
Y+ = phenotype present and stronger than C6
Y− = phenotype present and weaker than C6

### Even Chain Fatty Acids Rescue Inhibition of Mtb Growth in Cholesterol Media by C6

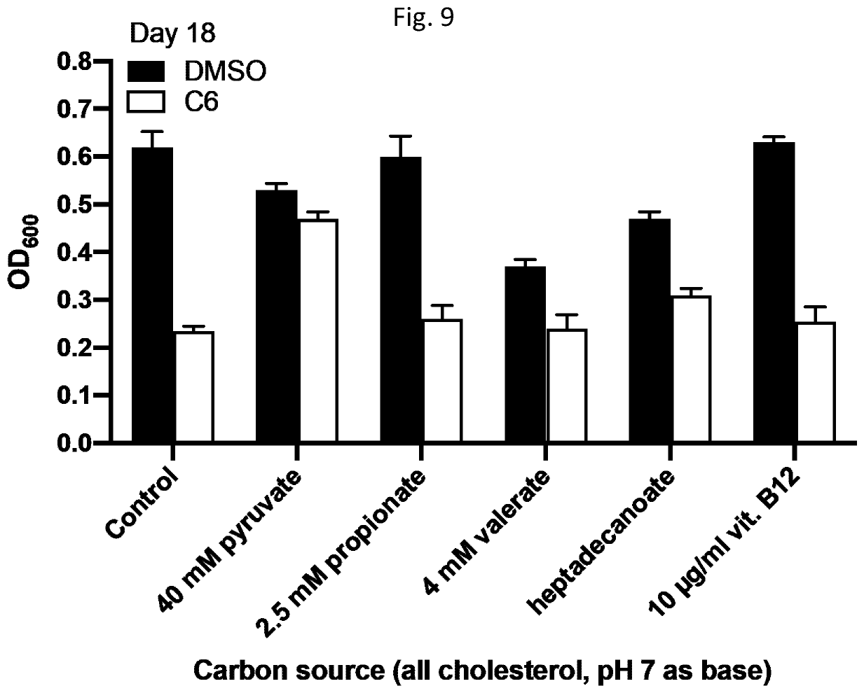

FIG. 8 demonstrate that even chain fatty acids rescue inhibition of Mtb growth in cholesterol media by C6 while FIG. 9 demonstrates that odd chain fatty acids do not rescue inhibition of Mtb growth. Additionally, vitamin B 12 does not rescue inhibition of Mtb growth in cholesterol media by C6 as well. These findings also reveal a potential intersection between Mtb response to Cl⁻ with its metabolism.

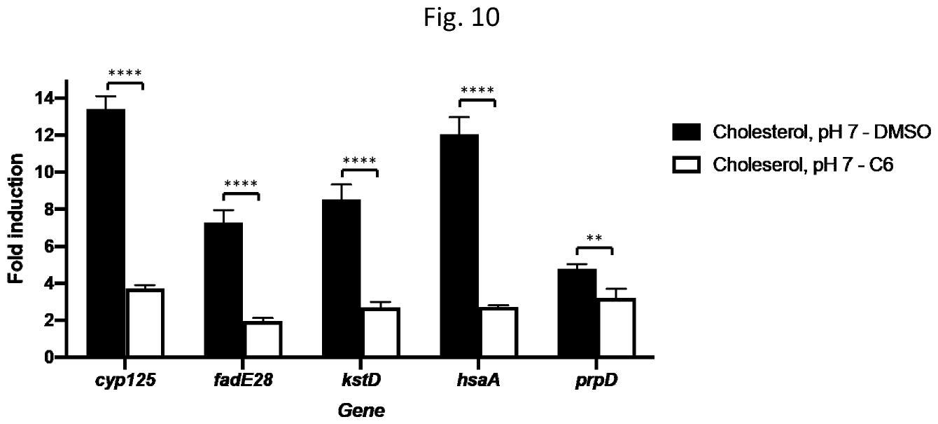

FIG. 10 demonstrates that C6 inhibits transcription of cholesterol utilization genes when Mtb is grown in cholesterol media. Expression for each of cyp125, fadE28, kstD, hsaA, and prpD was significantly reduced by C6.

### JSF-4538 Inhibits Mtb Growth In Vivo

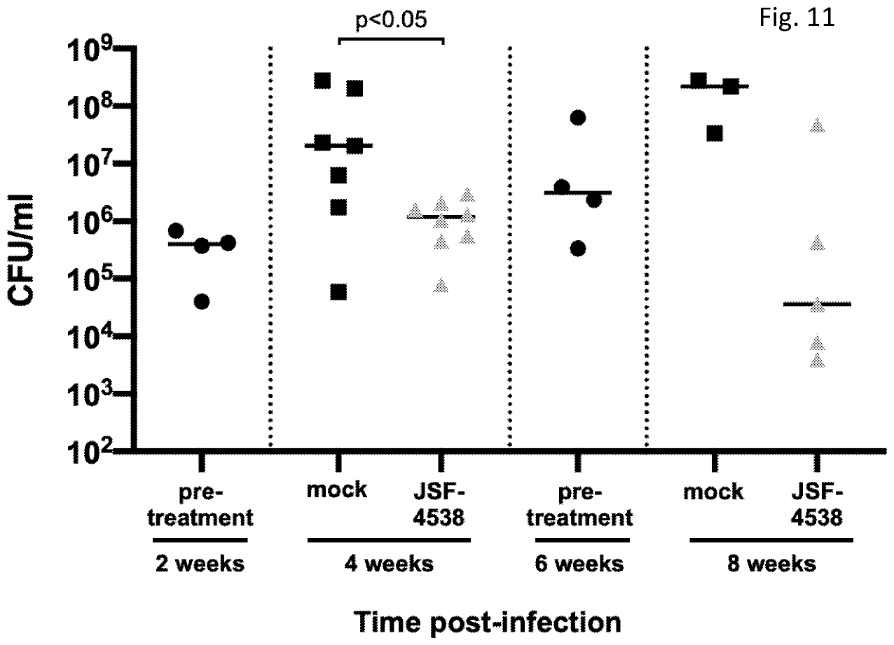

FIG. 11 demonstrates the in vivo activity of JSF-4538. C3HeB/FeJ WT mice were infected with Mtb for 2 or 6 weeks (pre-treatment), before mock-treatment or treatment with 150 mg/kg JSF-4538 (5 days/week via oral gavage) for a further 2 weeks. CFUs from lung homogenates plated at indicated times points post-infection are shown.

### Discussion

Successful colonization of the host by Mtb requires proper sensing and response to environmental cues for bacterial adaptation. In screening for compounds that perturb the response of Mtb to Cl⁻, we have uncovered a compound, C6, that inhibits Mtb host colonization in vivo. Our study thus illuminates how examination of Mtb response to an abundant ion like Cl⁻, an understudied facet of Mtb-host interactions that, however, has clear connections to other critical environmental cues and relevance to Mtb infection biology (MacGilvary et al, 2019; Tan et al, 2013), can be exploited in the discovery of compounds that disrupt Mtb response to environmental cues and host colonization.

Our SAR studies have begun to shed light on the structural components important for the phenotypes observed, and illustrate the correlation between perturbation of Cl⁻ response and inhibition of Mtb growth in host cells in a subset of compounds. The inhibition of Mtb growth in cholesterol by C6 suggests complexity in its mechanism of action that may extend beyond modulation of Mtb response to environmental cues. Of note however, a second compound with shared structural components to C6 that was also called as having antitubercular activity in cholesterol media in that assay set did not demonstrate dysregulation of Mtb Cl⁻ response in our original rv2390c′::GFP screen. This is in accord with our SAR results, as it lacks the 2-ethylthio group essential to date for our Cl⁻ response phenotype within this general class of compounds, and indicates that the structural components that drive the Cl⁻ response phenotype versus the cholesterol phenotype can be separated. Further studies will be required to tease this aspect apart, and to pursue the potential intersection between Mtb Cl⁻ response and its metabolism suggested by our unexpected finding that the rv2390c′::GFP reporter is induced in cholesterol media, with the presence of increased Cl⁻ levels additively increasing reporter signal.

We have also noted the structural similarity of C6 with compound V-58, which also inhibits Mtb growth in cholesterol, with the pyrimidine 5-position in both compounds linked by a methylene to a heterocycle (piperazine 4-position in C6 and piperidine 4-position for V-58) (Johnson et al, 2017). The remaining nitrogen of the piperazine in C6 and piperidine in V-58 are both attached to a nitrogen-containing heterocycle (Johnson et al, 2017). V-58 has been elegantly demonstrated to activate Rv1625c and thus promote cAMP synthesis (Johnson et al, 2017). While we have not initiated a further comparison of both compounds, we expect that future studies will delineate any mechanistic overlap between dysregulation of Mtb response to high [Cl⁻], activation of Rv1625c, and modulation of cAMP synthesis.

Reporter Mtb strains are unique and powerful tools that have provided insight into various aspects of Mtb biology in vitro and in vivo (Abramovitch et al, 2011; Abramovitch et al, 2018; Huang et al, 2018; Johnson et al, 2015; MacGilvary et al, 2019; MacGilvary et al, 2018; Sukumar et al, 2014; Tan et al, 2013; Zheng et al, 2017), and our study reinforces their utility in uncovering compounds that can serve as chemical probes for understanding Mtb environmental cue response, and with the potential for development as lead compounds. Additionally, analyses of the intrabacterial and intracellular accumulation of compounds represent approaches with broad applicability to study the general phenomenon of drug/chemical probe interactions within the cell. As shown here, they enable analysis of compound accumulation within the bacterium and/or host cells, independently or in the combined context of infection, revealing how differential accumulation may contribute to differences in compound antibacterial activity. These approaches are complementary to examples of intrabacterial drug metabolism (IBDM) from our lab and others (Awasthi et al, 2017; Li et al, 2015; Nixon et al, 2014; Wang et al, 2020; Wang et al, 2019).

Particularly given the growing understanding of the intimate connections between homeostasis and response to disparate ionic cues (MacGilvary et al, 2019), and the intersection of Mtb environmental response with its metabolism (Baker et al, 2019), we propose that this facet of Mtb-host interactions represents an area with meaningful potential in the development of new antituberculars, which may also exhibit synergy with current drugs. In addition, chemical probes identified in screens such as these provide new tools that can be exploited in further understanding the molecular mechanisms underlying Mtb response to a given environmental cue, providing critical insight into fundamental aspects of Mtb biology.

### Materials and Methods

### Mtb Strains and Culture

Mtb strains for bacterial growth and growth inhibition assays with J774 cells were in the CDCl551 background. The Mtb strains for primary murine bone marrow-derived macrophage infections and mouse infections were in the Erdman background. CDCl551 (rv2390c′::GFP) was used for the chemical compound screen and has been previously described (Tan et al, 2013). CDCl551 (rv1405c′::GFP) was generated in a similar manner, with a 744 bp region immediately upstream of the rv1405c open reading frame PCR amplified and cloned in front of GFPmut2 in the modified replicating plasmid pSE100, and the resultant plasmid transformed into Mtb. To generate the P1′::mKO construct, a codon-optimized monomeric Kusabira Orange (mKO) driven by the P1 promoter was introduced into the pDE43-MEK vector by Gateway cloning (Blumenthal et al, 2010; Huang et al, 2018). The reporter was then transformed into CDCl551, with selection on 7H10 agar containing 25 μg/ml kanamycin. Routine propagation of Mtb cultures were as previously described, in standing T25 flasks with filter caps, in 7H9 Middlebrook medium supplemented with OADC, 0.05% Tween 80, buffered at pH 7.0 with 100 mM MOPS (Abramovitch et al, 2011). 50 μg/ml hygromycin B or 25 μg/ml kanamycin was added as needed for maintenance of reporter plasmids. Preparation of Mtb stocks used for mice infections was as previously described (Sukumar et al, 2014).

Small Molecule Chemical Screen with Fluorescent Reporter Mtb Strain

The CDCl551 (rv2390c′::GFP) reporter Mtb strain was utilized for the small molecule compound screen. Screening compounds were from The Rockefeller University's High Throughput and Spectroscopy Center, and were aliquoted at 20 μM in 25 μl 7H9, pH 6.4, 250 mM NaCl media in bar-coded 384-well black, clear-bottom plates. Mid-log phase (OD₆₀₀˜0.6) reporter Mtb grown in standard 7H9, pH 7 media were resuspended in 7H9, pH 6.4, 250 mM NaCl media, and 25 μl of the bacterial suspension added to each well, at a final OD₆₀₀=0.05 (final compound concentration=10 μM), using a Janus Ministation (PerkinElmer). For each test plate, two columns of controls (16 wells each) containing DMSO as a carrier control were included, with one column consisting of Mtb in 7H9, pH 6.4 media (negative control), and the other column consisting of Mtb in 7H9, pH 6.4, 250 mM NaCl media (positive control). Bacteria in all control wells were also inoculated at a final OD₆₀₀=0.05. All wells (test and control) also contained 50 g/ml hygromycin B and 100 μg/ml cycloheximide. Plates were kept in humidified ziplock bags in a 37° C., 5% CO₂ incubator. GFP signal (top read monochromator, excitation 488 nm, emission 510 nm) and OD₆₀₀ were measured 6 days post-inoculation with a PerkinElmer EnVision multimode plate reader.

Validation tests of called hits and assays with the rv1405c′::GFP reporter Mtb strain were done as dose-response assays in triplicate runs, with compounds tested beginning at a final concentration of 20 μM and two-fold dilutions down to 0.039 μM.

### Liquid Chromatography-Mass Spectrometry Authentication of Screen Compounds

Compounds were dissolved in HPLC-grade DMSO to 5 mM and diluted 1:25 in HPLC-grade methanol. 2 μl samples were injected via the autosampler of an Agilent Infinity 1260 HPLC system, set to a flow rate of 200 l/minute. A gradient of 5%-90% acetonitrile in 0.1% formic acid was developed over 6 minutes on a ZORBAX Rapid Resolution HTExtend 1.8 μm C18 80 Å column (2.1×50 mm), with a 600 bar pressure limit. The column eluant was analyzed using an Agilent 6230 TOF Mass Spectrometer/PDA detector, and purity was determined by area under the curve (AUC) of total ion current and absorbance at 208 nm wavelength. Caffeine and DMSO alone were used as positive and negative controls respectively.

### J774 Cell Culture and Infection

For the tertiary screen of selected hit compounds in J774 murine macrophage-like cells, 30,000 J774 cells/well were seeded in bar-coded 384-well black, clear bottom plates in a 30 μl volume in J774 infection media (DMEM+10% FBS+1 mM sodium pyruvate+2 mM L-glutamine) using a ThermoFisher Multidrop Combi reagent dispenser, one day prior to assay start. On the day of the assay, log-phase Mtb constitutively expressing mKO were pelleted and resuspended in 1 ml basal uptake buffer (0.5% bovine serum albumin+25 mM dextrose+0.5 mM MgCl₂+1 mM CaCl₂+0.1% gelatin in phosphate buffered saline (PBS), passed 6× though a tuberculin syringe with a 25 G×5/8″ needle, then resuspended in J774 infection media to a final OD₆₀₀=0.2. 10 μl of this bacterial suspension was added to each well of the 384-well plates containing the J774 cells, followed by addition of 10 μl of the appropriate compounds (pre-aliquoted in v-bottom plates in a 1:1 mix of PBS:J774 infection media). Addition of Mtb and compounds were conducted using a Janus Ministation. For each test plate, two columns of controls (16 wells each) were included, with one column consisting of treatment with DMSO as a carrier control (negative control), and the other column consisting of treatment with 5 μM rifampicin (positive control). This tertiary assay was done as a dose-response assay in duplicate runs, with compounds tested beginning at a final concentration of 20 μM and two-fold dilutions down to 0.039 μM. Plates were kept in humidified ziplock bags in a 37° C., 5% CO₂ incubator. 6 days post-infection, mKO fluorescence signal was read using a PerkinElmer EnVision multimode plate reader (bottom read filter, excitation 530/8 nm, Bodipy TMR D555 single mirror, emission 579/25 nm). mKO fluorescence in DMSO-treated control samples were defined as 100% growth, and fluorescence observed in treatment conditions calculated as a percentage from that set value.

For J774 infection in 96-well plates, 180,000 J774 cells were seeded per well in 180 μl of J774 infection media one day prior to infection. On the day of the assay, log-phase Mtb constitutively expressing mKO were pelleted and resuspended in 1 ml basal uptake buffer, passed 6× though a tuberculin syringe with a 25 G×5/8″ needle, then resuspended in J774 infection media to a final OD₆₀₀=0.2. Media was removed from 96-well plate containing the J774 cells, with 140 μl of fresh J774 infection media added back to each well. 40 μl of the Mtb suspension at OD₆₀₀=0.2 was added to each well, followed by addition of appropriate concentration of compound or DMSO in a 20 μl final volume in J774 infection media (compound final concentration 10 μM, rifampicin final concentration 5 μM, DMSO final concentration 0.1%/well). Bacterial mKO fluorescence was read immediately after infection using a Biotek Synergy Neo2 microplate reader (bottom read monochromator, excitation 543/10 nm, emission 565/10 nm), and again 6 days post-infection. Plates were kept in humidified ziplock bags in a 37° C., 5% CO₂ incubator. mKO fluorescence in DMSO-treated control samples were defined as 100% growth, and fluorescence observed in treatment conditions calculated as a percentage from that set value.

### Primary Murine Bone Marrow-Derived Macrophage Culture and Infection

C57BL/6J wild type mice (Jackson Laboratories) were used for extraction of bone marrow-derived macrophages. Cells were harvested and expanded in DMEM containing 10% FBS, 15% L-cell conditioned media, 2 mM L-glutamine, 1 mM sodium pyruvate, and penicillin/streptomycin as needed in a 37° C. incubator in a 5% CO₂ atmosphere. Macrophage infections were performed essentially as previously described (Abramovitch et al, 2011), with the addition of 10 μM compound C6, or DMSO as a carrier control, to the media 2 hours after bacterial inoculation, after non-internalized Mtb had been washed away. Macrophages were lysed with water containing 0.01% sodium dodecyl sulfate and a dilution series of the samples plated on 7H10 agar for CFU determination.

### Reporter Mtb Strain Broth Assays

Broth culture assays with the CDCl551 (rv2390c′::GFP) reporter Mtb strain was carried out essentially as previously described, in standing filter-cap T-25 flasks in 10 ml of 7H9 or cholesterol media buffered at pH 7, with addition of 250 mM NaCl as needed (MacGilvary et al, 2019; Tan et al, 2013). Cholesterol medium consisted of 7H9 broth supplemented with 0.5 g/l fatty acid-free bovine serum albumin, 14.5 mM NaCl, 0.2 mM cholesterol, and 0.1% tyloxapol, buffered to pH 7.0 with 100 mM MOPS (Smith et al, 2020). Cholesterol stocks were prepared at 100 mM, in 1:1 ethanol:tyloxapol (Lee et al, 2013; Smith et al, 2020; VanderVen et al, 2015). In brief for the reporter assay, log-phase Mtb was used to inoculate 10 ml of fresh 7H9 or cholesterol media at pH 7, or 7H9 or cholesterol media at pH 7 supplemented with 250 mM NaCl media, at a starting OD₆₀₀=0.05, with 50 μg/ml hygromycin B added to maintain reporter plasmid selection. DMSO as a carrier control or 10 μM of compound to be tested was added at the start of the assay as appropriate. Mtb was fixed at indicated time points in 4% paraformaldehyde (PFA) in PBS. Analysis of bacterial fluorescence was performed using a BD FACSCalibur, and data analyzed using FlowJo (BD Biosciences). Initial follow up tests of C5 and C6 utilized commercially obtained compounds from Molport, SIA (Riga, Latvia).

### Physiochemical and ADME Characterization of Compounds

Assays examining mouse and human liver microsomal stability, kinetic aqueous solubility, mouse and human plasma protein binding and stability, and human cytochrome P450 inhibition characteristics of the compounds were run by BioDuro, Incorporated, using standard protocols as previously described (Wang et al, 2020). hERG inhibition assays were also performed by BioDuro, Incorporated. For this assay, HEK-293 cells stably expressing the hERG K⁺ channel were provided by the Institute of SARL (CreaCell). Cells were grown in DMEM supplemented with 10% FBS and 0.8 mg/ml G418. For electrophysiology experiments, the cells were continuously superfused by extracellular saline with 140 mM NaCl, 3.5 mM KCl, 1 mM MgCl₂, 2 mM CaCl₂, 10 mM dextrose, 10 mM HEPES, and 1.25 mM NaH₂PO₄, pH 7.4. Compounds for the assay were dissolved in DMSO at a concentration of 10 mM for cisapride, a positive control, and 30 mM for C6, and diluted in extracellular saline to the appropriate concentration (1 nM-1 μM for cisapride and 0.3-30 μM for C6). The glass micropipettes for whole-cell patch-clamp recording were filled with intracellular saline containing 20 mM KCl, 115 mM potassium L-aspartate, 1 mM MgCl₂, 5 mM EGTA, 10 mM HEPES, 2 mM Na₂-ATP, pH 7.2.

The hERG current was measured at a holding potential of −80 mV and then depolarized to −50 mV for 0.5 s to test the leak current. The voltage was then depolarized to 30 mV for 2.5 s. The peak tail current was induced by a repolarizing pulse to −50 mV for 4 s. An inter-pulse interval of 10 s enabled recovery from inactivation to quantify the effect of the test article on the hERG tail current. The test compound was then added to the investigated cell from a nearby capillary, typically for another three mins per concentration. The data were collected by EPC-10 amplifier and stored in PatchMaster (HEKA) software. Current amplitude values were plotted versus time to illustrate the effect of the compound and quantify the percentage of channel inhibition. The results obtained from at least three cells were pooled and fitted with a non-linear regression to estimate the mean IC₅₀ value.

### Pharmacokinetic Assays

Two female CD-1 mice (Charles River Laboratories) were dosed once with C5 or C6 administered orally at 5 or 25 mg/kg in 5% dimethylacetamide/60% polyethylene glycol 300/35% D5W (5% dextrose in water). Blood samples were collected in K₂EDTA coated tubes pre-dose, 0.5, 1, 3 and 5 hrs post-dose, placed on ice, and centrifuged to recover plasma. The recovered plasma was kept at −80° C. as needed prior to analysis, which was performed as previously described (Wang et al, 2020). In brief, a Sciex Applied Biosystems Qtrap 4000 triple-quadrupole mass spectrometer coupled to an Agilent 1260 HPLC system was used for HPLC coupled to tandem mass spectrometry (LC-MS/MS) quantitative analysis of the samples, with chromatography attained using an Agilent Zorbax SB-C8 column (2.1×30 mm; particle size, 3.5 μm) and a reverse phase gradient elution. The aqueous mobile phase consisted of Milli-Q deionized water with 0.1% formic acid (A), while the organic mobile phase was 0.1% formic acid in acetonitrile (B). The gradient set-up was as follows: 5-90% B over 2 mins, 1 min at 90% B, then an immediate drop to 5% B and an additional 1 min at 5% B. Quantification of molecules was carried out via multiple-reaction monitoring of parent/daughter transitions in electrospray positive-ionization mode, and sample analyses accepted if quality control and standard samples had concentrations within 20% of the nominal concentration. Preparation of compounds for generation of standard curves and quality control spiking solutions were performed as previously described (Wang et al, 2020). Data were processed using Analyst software (Version 1.6.2; Applied Biosystems Sciex).

### Vero Cell Toxicity Assay

Vero cells (African green monkey kidney epithelial cells; ATCC CCL-81) were maintained in DMEM+10% FBS. For the cell toxicity assay, Vero cells were plated at 1×10⁵ cells/well in a 96-well plate and incubated for 2-3 h to allow cells to settle. The test compound was dissolved in DMSO at a final concentration of 12 mg/ml. The test compound solution was then added to plated cells, resulting in final test concentrations of 50 to 0.78 μg/ml, with each concentration tested at least in duplicate. To evaluate bacterial cell viability, 20 μl of a 1:20 MTS:PMS reagent (Promega CellTiter 96 AQueous non-radioactive cell proliferation assay kit) was added to each well after 72 h of incubation at 37° C. and the plate then incubated for an additional 3 h. Absorbance at 490 nm was read on a Molecular Devices SpectraMax M5 microplate reader. The CC₅₀ (the minimal amount of compound that inhibited Vero cell growth by 50%) was extrapolated by plotting absorbance at 490 nm versus the concentration of untreated Vero cells in control plates.

### Mtb Growth Assays

Mtb grown to log-phase in 7H9 medium, pH 7.0 was used to inoculate 10 ml 7H9 medium, pH 7.0 containing 10 μM C6/C6 analogs or DMSO as a carrier control in standing, filter-cap T25 flasks, at a starting OD₆₀₀ of 0.05. Bacterial growth was tracked by measuring OD₆₀₀ every 3 days for 12 days. For growth assays in cholesterol medium, log-phase Mtb grown in 7H9 medium, pH 7.0 was used to inoculate 10 ml of cholesterol medium containing 10 μM C6/C6 analog, or DMSO as a carrier control, in standing, vented T25 flasks, at a starting OD₆₀₀ of 0.05. Bacterial growth was tracked by measurement of D₆₀₀ every 3 days over 12 days.

### Phagosomal Maturation Assays

Carboxyfluorescein (pH readout), DQ-BSA/Alexa Fluor 594 (AF594) (proteolysis readout), and 10,10′-Bis[3-carboxylpropyl]-9,9′-biacridinium (BAC)/AF594 (Cl⁻ readout) beads were generated as previously described (Podinovskaia et al, 2013; Tan et al, 2013; Tan et al, 2017; Yates et al, 2008). Bone marrow-derived macrophages were isolated from C57BL/6J mice (Jackson Laboratories) and maintained as described above. 2×10⁵ macrophages/well were seeded into 96-well clear bottom black plates, and utilized in assays one-two days post-seeding. Macrophages were first washed 3 times with pre-warmed assay buffer, with fresh buffer containing 10 μM C6, or DMSO as a carrier control, then added back to each well as appropriate. Assay buffer consisted of PBS, pH 7.2, supplemented with 5% FBS, 5 mM dextrose, 1 mM CaCl₂, 2.7 mM KCl, and 0.5 mM MgCl₂ in the case of assays with the carboxyfluorescein or DQ-BSA/AF594 beads (Buter et al, 2019; Dutta et al, 2020). For assays with the BAC/AF594 beads, assay buffer consisted of PBS, pH 7.2, supplemented with 5% FBS, 5 mM dextrose, 1 mM calcium acetate, 1.35 mM K₂SO₄, and 0.5 mM MgSO₄ (Tan et al, 2013). Sensor beads at ˜2-5 beads/macrophage in appropriate assay buffer were finally added, with data acquisition on a Biotek Synergy H1 microplate reader with bottom read signal detection started within 2-3 minutes of bead addition. Excitation/emission wavelengths were 450 nm/520 nm and 490 nm/520 nm for carboxyfluorescein, 490 nm/520 nm for DQ-BSA, 365 nm/505 nm for BAC, and 590 nm/617 nm for AF594. Each experiment was run with a total of 3-5 replicate wells/condition, with temperature maintained at 37° C. throughout the assay. Wells were read every 2 minutes for 2 hours for the carboxyfluorescein bead assays, every 2 minutes for 4 hours for the DQ-BSA/AF594 bead assays, and every 1 minute for 2 hours for the BAC/AF594 bead assays. Analyses of the results were performed as previously described (Buter et al, 2019; Dutta et al, 2020; Podinovskaia et al, 2013; Tan et al, 2013).

### Intrabacterial and Intracellular Drug Accumulation Assays

Each experimental run for these assays was carried out with 2-3 samples. For intrabacterial drug accumulation assays, Mtb CDCl551 was grown in 10 ml of 7H9, pH 7 medium in standing filter-cap T-25 flasks to log phase (OD₆₀₀˜0.6), and the cultures treated with various concentrations of C6 or indicated C6 analogs, or DMSO as a carrier control, for 24 h. Samples were then pelleted, resuspended in 1 ml of pre-chilled extraction buffer (2:2:1 acetonitrile:methanol:water), transferred to a 2 ml screw-cap tube containing 0.5 ml of 0.1 mm zirconia/silica beads (BioSpec Products, Inc), and stored at −80° C. prior to further processing. J774 murine macrophage-like cells were maintained in J774 infection media, and seeded at 1×10⁶ cells/well in 6-well plates for ICDM, and 2×10⁷ cells/flask in T-75 flasks for assays examining drug accumulation during Mtb infection of J774 cells. For intracellular drug accumulation studies, the assay was initiated 1 h after cell seeding, by treating the cells with C6 or C6 analogs at various concentrations, or DMSO as a carrier control, for 24 h. After the treatment period, the cells were washed 3× with cold PBS, before 1 ml of cold PBS was added to each well and the cells scraped and collected. Samples were pelleted, resuspended in 1 ml of pre-chilled extraction buffer, and stored at −80° C. prior to further processing. For assays with infected J774 cells, infection of the J774 cells one day after seeding was essentially as previously described (Liu et al, 2016). In brief, the J774 cells were infected with log-phase Mtb CDCl551 at a MOI=5 for 2 h, before media removed and replaced with fresh pre-warmed media. Media was changed daily, and treatment with C6 or C6 analogs at various concentrations, or DMSO as a carrier control, initiated 5 days post-infection. 24 h after initiation of treatment, cells were washed twice with media, and 5 ml of cold PBS added. Cells were then scrapped and the samples pelleted, before resuspension in 1 ml of pre-chilled extraction buffer. Samples were then transferred to a 2 ml screw-cap tube containing 0.5 ml of 0.1 mm zirconia/silica beads and stored at −80° C. prior to further processing.

For continued processing, samples were thawed and lysed by bead beating for the Mtb and Mtb infected J774 assays (6×45 s, 6.5 m/s, placing samples on ice for 5 minutes in between each bead beating cycle), or by 4 freeze-thaw cycles for the uninfected J774 cells (5 min freeze step in a dry ice/ethanol bath, 30 s thaw step in water, with 10 s vortexing between cycles). After lysis, the samples were centrifuged at 13,000 rpm, 10 min, 4° C., and the supernatant then filtered through a 0.22 m Spin-X centrifuge tube filter (Corning Costar) (13,000 rpm, 10 min, 4° C.). Samples were stored at −80° C. until LC-MS analysis.

For LC-MS analysis, the samples were chromatographed using a Chromolith SpeedROD column with a gradient of water and acetonitrile acidified with 0.1% formic acid, and then analyzed using an Agilent 1260 liquid chromatography system coupled to an Agilent 6120 quadrupole mass spectrometer. The mass resolution ranged from 10 to 2000 with an accuracy of ±0.13 Da within the calibrated mass range in scan mode. Drug metabolite concentrations were calculated using the method of standard addition with authentic chemical standards. Signal intensity of each experimental compound was quantified by standard curve for an authentic/independently synthesized sample, and normalized by the cell number.

### Mouse Mtb Infections

C3HeB/FeJ wild type mice (Jackson Laboratories) were infected intranasally with 10³ CFUs of Mtb (35 μl), under light anesthesia with 2% isoflurane (MacGilvary et al, 2019; Sukumar et al, 2014; Tan et al, 2013). Compound C6 was prepared for treatment by suspending an appropriate amount of C6 (31-35 mg/ml as needed for final dosage in mice at 250 mg/kg) in Ultrapure water (Invitrogen) containing 0.5% carboxymethyl cellulose (CMC) and 0.5% Tween 80, and stirring the mixture overnight at 4° C. The next day the suspension was homogenized by bead beating with 5 mm steel beads in a TOMY microtube mixer (2×20 s), and stored at 4° C. until use. C6 suspension for in vivo use was used within two weeks of preparation. The mice were treated by oral gavage five time per week for two or 4 weeks with 250 mg/kg C6 in 0.5% CMC+0.5% Tween 80, or mock-treated with 0.5% CMC+0.5% Tween 80 as a vehicle control (200 μl volume). Administration began at two or six weeks post-infection as indicated. At time of sacrifice, the left lobe and accessory right lobe of the lung were homogenized in PBS+0.05% Tween 80 and serial dilutions plated on 7H10 agar plates with 100 μg/ml cycloheximide for bacterial load determination. The remaining three right lobes were fixed in 4% PFA in PBS. One lobe was used for histological analysis by standard hematoxylin and eosin (H&E) staining (Tufts Comparative Pathology Services). A Nikon Eclipse E400 with a SPOT insight color digital camera was used to image the histology samples.

Resources utilized are presented in Table 4.

TABLE 4
Resources used.

| | | |
|---|---|---|
| REAGENT or RESOURCE | SOURCE | IDENTIFIER |

Bacterial Strains

| | | |
|---|---|---|
| M. tuberculosis CDC1551 | Lab stock | BEI NR-13649 |
| M. tuberculosis CDC1551(rv2390c′::GFP) | Tan et al., 2013 | N/A |
| M. tuberculosis CDC1551(rv1405c′::GFP) | This paper | N/A |
| M. tuberculosis CDC1551(P1′::mKO) | This paper | N/A |
| M. tuberculosis Erdman | Lab stock | ATCC 35801 |
| E. coli chemically competent TOP10 | ThermoFisher | Cat# C404003 |

Chemicals, Peptides, and Recombinant Proteins

| | | |
|---|---|---|
| C6 | Molport Inc. | Cat# MolPort-016- |
| | | 629-492 |
| Small molecules labelled as JSF-# | This paper | N/A |
| Screening compounds | Rockefeller | N/A |
| | University High | |
| | Throughput and | |
| | Spectroscopy Center | |
| Rifampicin | TCI America | Fisher Scientific |
| | | Cat# R007925G |
| 5-(and-6)-carboxyfluorescein succinimidyl ester, | ThermoFisher | Cat# C1311 |
| mixed isomers | | |
| DQ Green BSA | ThermoFisher | Cat# D12050 |
| Alexa Fluor 594 NHS ester (succinimidyl ester) | ThermoFisher | Cat# A20004 |
| 10,10′-Bis[3-carboxylpropyl]-9,9′-acridinium | Emp Biotech | Cat# AF-0406- |
| dinitrate di-NHS ester (BAC-SE) | | D005.0-001 |

Critical Commercial Assays

| | | |
|---|---|---|
| CellTiter 96 AQᵤₑₒᵤₛ non-radioactive cell | Promega | Cat# G5421 |
| proliferation assay kit | | |

Experimental Models: Cell Lines

| | | |
|---|---|---|
| Mouse: J774A.1 cells | ATCC | ATCC TIB-67 |
| Monkey: Vero cells | ATCC | ATCC CCL-81 |

Experimental Models: Organisms/Strains

| | | |
|---|---|---|
| Mouse: C57BL/6J | The Jackson | JAX: 000664 |
| | Laboratory | |
| Mouse: C3HeB/FeJ | The Jackson | JAX: 000658 |
| | Laboratory | |
| Mouse: CD-1 IGS | Charles River | Crl: CD1(ICR), |
| | Laboratories | strain code 022 |

Oligonucleotides

| | | |
|---|---|---|
| Primer: rv1405c promoter forward: 5′ CCA TCC | This paper | N/A |
| AGC GTG GTC GAT AGC A (SEQ ID NO: 1) | | |
| Primer: rv1405c promoter reverse: 5′ CAG GTC | This paper | N/A |
| TCC TGA GAA GTA AGT GAT GTG GC (SEQ | | |
| ID NO: 2) | | |

Recombinant DNA

| | | |
|---|---|---|
| Plasmid: rv2390c′::GFP | Tan et al., 2013 | N/A |
| Plasmid: rv1405c′::GFP | This paper | N/A |
| Plasmid: Pl′::mKO | This paper | N/A |

Software and Algorithms

| | | |
|---|---|---|
| FlowJo | BD | https://www. |
| | | flowjo.com |
| Graphpad Prism | GraphPad Software, | https://www.graph |
| | Inc. | pad.com/scientific- |
| | | software/prism/ |

### REFERENCES

Abramovitch, R. B. (2018). Mycobacterium tuberculosis reporter strains as tools for drug discovery and development. IUBMB Life 70, 818-825.
Abramovitch, R. B., Rohde, K. H., Hsu, F. F., and Russell, D. G. (2011). aprABC: a Mycobacterium tuberculosis complex-specific locus that modulates pH-driven adaptation to the macrophage phagosome. Mol Microbiol 80, 678-694.
Awasthi, D., and Freundlich, J. S. (2017). Antimycobacterial metabolism: illuminating Mycobacterium tuberculosis biology and drug discovery. Trends Microbiol 25, 756-767.
Baker, J. J., Dechow, S. J., and Abramovitch, R. B. (2019). Acid fasting: modulation of Mycobacterium tuberculosis metabolism at acidic pH. Trends Microbiol 27, 942-953.
Bhaskar, A., Chawla, M., Mehta, M., Parikh, P., Chandra, P., Bhave, D., Kumar, D., Carroll, K. S., and Singh, A. (2014). Reengineering redox sensitive GFP to measure mycothiol redox potential of Mycobacterium tuberculosis during infection. PLoS Pathog 10, e1003902.
Blumenthal, A., Trujillo, C., Ehrt, S., and Schnappinger, D. (2010). Simultaneous analysis of multiple Mycobacterium tuberculosis knockdown mutants in vitro and in vivo. PLoS One 5, e15667.
Buter, J., Cheng, T. Y., Ghanem, M., Grootemaat, A. E., Raman, S., Feng, X., Plantijn, A. R., Ennis, T., Wang, J., Cotton, R. N., et al. (2019). Mycobacterium tuberculosis releases an antacid that remodels phagosomes. Nat Chem Biol 15, 889-899.
Converse, P. J., Karakousis, P. C., Klinkenberg, L. G., Kesavan, A. K., Ly, L. H., Allen, S. S., Grosset, J. H., Jain, S. K., Lamichhane, G., Manabe, Y. C., et al. (2009). Role of the dosR-dosS two-component regulatory system in Mycobacterium tuberculosis virulence in three animal models. Infect Immun 77, 1230-1237.
Driver, E. R., Ryan, G. J., Hoff, D. R., Irwin, S. M., Basaraba, R. J., Kramnik, I., and Lenaerts, A. J. (2012). Evaluation of a mouse model of necrotic granuloma formation using C3HeB/FeJ mice for testing of drugs against Mycobacterium tuberculosis. Antimicrob Agents Chemother 56, 3181-3195.
Dutta, N. K., Bruiners, N., Zimmerman, M. D., Tan, S., Dartois, V., Gennaro, M. L., and Karakousis, P. C. (2020). Adjunctive host-directed therapy with statins improves tuberculosis-related outcomes in mice. J Infect Dis 221, 1079-1087.
Gautam, U. S., McGillivray, A., Mehra, S., Didier, P. J., Midkiff, C. C., Kissee, R. S., Golden, N. A., Alvarez, X., Niu, T., Rengarajan, J., et al. (2015). DosS Is required for the complete virulence of Mycobacterium tuberculosis in mice with classical granulomatous lesions. Am J Respir Cell Mol Biol 52, 708-716.
Harper, J., Skerry, C., Davis, S. L., Tasneen, R., Weir, M., Kramnik, I., Bishai, W. R., Pomper, M. G., Nuermberger, E. L., and Jain, S. K. (2012). Mouse model of necrotic tuberculosis granulomas develops hypoxic lesions. J Infect Dis 205, 595-602.
Huang, L., Kushner, N. L., Theriault, M. E., Pisu, D., Tan, S., McNamara, C. W., Petrassi, H. M., Russell, D. G., and Brown, A. C. (2018a). The deconstructed granuloma: a complex high-throughput drug screening platform for the discovery of host-directed therapeutics against tuberculosis. Front Cell Infect Microbiol 8, 275.
Huang, L., Nazarova, E. V., Tan, S., Liu, Y., and Russell, D. G. (2018b). Growth of Mycobacterium tuberculosis in vivo segregates with host macrophage metabolism and ontogeny. J Exp Med 215, 1135-1152.
Inoyama, D., Paget, S. D., Russo, R., Kandasamy, S., Kumar, P., Singleton, E., Occi, J., Tuckman, M., Zimmerman, M. D., Ho, H. P., et al. (2018). Novel pyrimidines as antitubercular agents. Antimicrob Agents Chemother 62, e02063-17.
Irwin, S. M., Driver, E., Lyon, E., Schrupp, C., Ryan, G., Gonzalez-Juarrero, M., Basaraba, R. J., Nuermberger, E. L., and Lenaerts, A. J. (2015). Presence of multiple lesion types with vastly different microenvironments in C3HeB/FeJ mice following aerosol infection with Mycobacterium tuberculosis. Dis Model Mech 8, 591-602.
Irwin, S. M., Prideaux, B., Lyon, E. R., Zimmerman, M. D., Brooks, E. J., Schrupp, C. A., Chen, C., Reichlen, M. J., Asay, B. C., Voskuil, M. I., et al. (2016). Bedaquiline and pyrazinamide treatment responses are affected by pulmonary lesion heterogeneity in Mycobacterium tuberculosis infected C3HeB/FeJ mice. ACS Infect Dis 2, 251-267.
Johnson, B. K., Colvin, C. J., Needle, D. B., Mba Medie, F., Champion, P. A., and Abramovitch, R. B. (2015). The carbonic anhydrase inhibitor ethoxzolamide inhibits the Mycobacterium tuberculosis PhoPR regulon and Esx-1 secretion and attenuates virulence. Antimicrob Agents Chemother 59, 4436-4445.
Johnson, R. M., Bai, G., DeMott, C. M., Banavali, N. K., Montague, C. R., Moon, C., Shekhtman, A., VanderVen, B., and McDonough, K. A. (2017). Chemical activation of adenylyl cyclase Rv1625c inhibits growth of Mycobacterium tuberculosis on cholesterol and modulates intramacrophage signaling. Mol Microbiol 105, 294-308.
Lanoix, J. P., Betoudji, F., and Nuermberger, E. (2016a). Sterilizing activity of pyrazinamide in combination with first-line drugs in a C3HeB/FeJ mouse model of tuberculosis.
Antimicrob Agents Chemother 60, 1091-1096.
Lanoix, J. P., Ioerger, T., Ormond, A., Kaya, F., Sacchettini, J., Dartois, V., and Nuermberger, E. (2016b). Selective inactivity of pyrazinamide against tuberculosis in C3HeB/FeJ mice is best explained by neutral pH of caseum. Antimicrob Agents Chemother 60, 735-743.
Lanoix, J. P., Lenaerts, A. J., and Nuermberger, E. L. (2015). Heterogeneous disease progression and treatment response in a C3HeB/FeJ mouse model of tuberculosis. Dis Model Mech 8, 603-610.
Lee, W., VanderVen, B. C., Fahey, R. J., and Russell, D. G. (2013). Intracellular Mycobacterium tuberculosis exploits host-derived fatty acids to limit metabolic stress. J Biol Chem 288, 6788-6800.
Lenaerts, A., Barry, C. E., 3rd, and Dartois, V. (2015). Heterogeneity in tuberculosis pathology, microenvironments and therapeutic responses. Immunol Rev 264, 288-307.
Li, S. G., Vilcheze, C., Chakraborty, S., Wang, X., Kim, H., Anisetti, M., Ekins, S., Rhee, K. Y., Jacobs, W. R., Jr., and Freundlich, J. S. (2015). Evolution of a thienopyrimidine antitubercular relying on medicinal chemistry and metabolomics insights. Tetrahedron Lett 56, 3246-3250.
Liu, Y., Tan, S., Huang, L., Abramovitch, R. B., Rohde, K. H., Zimmerman, M. D., Chen, C., Dartois, V., VanderVen, B. C., and Russell, D. G. (2016). Immune activation of the host cell induces drug tolerance in Mycobacterium tuberculosis both in vitro and in vivo. J Exp Med 213, 809-825.
MacGilvary, N. J., Kevorkian, Y. L., and Tan, S. (2019). Potassium response and homeostasis in Mycobacterium tuberculosis modulates environmental adaptation and is important for host colonization. PLoS Pathog 15, e1007591.
MacGilvary, N. J., and Tan, S. (2018). Fluorescent Mycobacterium tuberculosis reporters: illuminating host-pathogen interactions. Pathog Dis 76. Martin, C., Williams, A., Hernandez-Pando, R., Cardona, P. J., Gormley, E., Bordat, Y., Soto, C. Y., Clark, S. O., Hatch, G. J., Aguilar, D., et al. (2006). The live Mycobacterium tuberculosis phoP mutant strain is more attenuated than BCG and confers protective immunity against tuberculosis in mice and guinea pigs. Vaccine 24, 3408-3419.
Mattila, J. T., Ojo, O. O., Kepka-Lenhart, D., Marino, S., Kim, J. H., Eum, S. Y., Via, L. E., Barry, C. E., 3rd, Klein, E., Kirschner, D. E., et al. (2013). Microenvironments in tuberculous granulomas are delineated by distinct populations of macrophage subsets and expression of nitric oxide synthase and arginase isoforms. J Immunol 191, 773-784.
Mehra, S., Foreman, T. W., Didier, P. J., Ahsan, M. H., Hudock, T. A., Kissee, R., Golden, N. A., Gautam, U. S., Johnson, A. M., Alvarez, X., et al. (2015). The DosR regulon modulates adaptive immunity and is essential for Mycobacterium tuberculosis persistence. Am J Respir Crit Care Med 191, 1185-1196.
Nixon, M. R., Saionz, K. W., Koo, M. S., Szymonifka, M. J., Jung, H., Roberts, J. P., Nandakumar, M., Kumar, A., Liao, R., Rustad, T., et al. (2014). Folate pathway disruption leads to critical disruption of methionine derivatives inMycobacterium tuberculosis. Chem Biol 21, 819-830.
Osada, N., Kohara, A., Yamaji, T., Hirayama, N., Kasai, F., Sekizuka, T., Kuroda, M., and Hanada, K. (2014). The genome landscape of the African green monkey kidney-derived vero cell line. DNA Res 21, 673-683.
Perez, E., Samper, S., Bordas, Y., Guilhot, C., Gicquel, B., and Martin, C. (2001). An essential role for phoP in Mycobacterium tuberculosis virulence. Mol Microbiol 41, 179-187. Perryman, A. L., Patel, J. S., Russo, R., Singleton, E., Connell, N., Ekins, S., and Freundlich, J. S. (2018). Naïve bayesian models for vero cell cytotoxicity. Pharm Res 35, 170.
Pethe, K., Swenson, D. L., Alonso, S., Anderson, J., Wang, C., and Russell, D. G. (2004). Isolation of Mycobacterium tuberculosis mutants defective in the arrest of phagosome maturation. Proc Natl Acad Sci USA 101, 13642-13647.
Podinovskaia, M., Lee, W., Caldwell, S., and Russell, D. G. (2013). Infection of macrophages with Mycobacterium tuberculosis induces global modifications to phagosomal function. Cell Microbiol 15, 843-859.
Rohde, K. H., Abramovitch, R. B., and Russell, D. G. (2007). Mycobacterium tuberculosis invasion of macrophages: linking bacterial gene expression to environmental cues. Cell Host Microbe 2, 352-364.
Smith, T. C., 2nd, Pullen, K. M., Olson, M. C., McNellis, M. E., Richardson, I., Hu, S., Larkins-Ford, J., Wang, X., Freundlich, J. S., Ando, D. M., et al. (2020). Morphological profiling of tubercle bacilli identifies drug pathways of action. Proc Natl Acad Sci USA 117, 18744-18753.
Sonawane, N. D., Thiagarajah, J. R., and Verkman, A. S. (2002). Chloride concentration in endosomes measured using a ratioable fluorescent Cl⁻ indicator: evidence for chloride accumulation during acidification. J Biol Chem 277, 5506-5513.
Sturgill-Koszycki, S., Schaible, U. E., and Russell, D. G. (1996). Mycobacterium-containing phagosomes are accessible to early endosomes and reflect a transitional state in normal phagosome biogenesis. EMBO J 15, 6960-6968.
Sturgill-Koszycki, S., Schlesinger, P. H., Chakraborty, P., Haddix, P. L., Collins, H. L., Fok, A. K., Allen, R. D., Gluck, S. L., Heuser, J., and Russell, D. G. (1994). Lack of acidification in Mycobacterium phagosomes produced by exclusion of the vesicular proton-ATPase. Science 263, 678-681.
Sukumar, N., Tan, S., Aldridge, B. B., and Russell, D. G. (2014). Exploitation of Mycobacterium tuberculosis reporter strains to probe the impact of vaccination at sites of infection. PLoS Pathog 10, e1004394.
Tan, S., Sukumar, N., Abramovitch, R. B., Parish, T., and Russell, D. G. (2013). Mycobacterium tuberculosis responds to chloride and pH as synergistic cues to the immune status of its host cell. PLoS Pathog 9, e1003282.
Tan, S., Yates, R. M., and Russell, D. G. (2017). Mycobacterium tuberculosis: readouts of bacterial fitness and the environment within the phagosome. Methods Mol Biol 1519, 333-347.
VanderVen, B. C., Fahey, R. J., Lee, W., Liu, Y., Abramovitch, R. B., Memmott, C., Crowe, A. M., Eltis, L. D., Perola, E., Deininger, D. D., et al. (2015). Novel inhibitors of cholesterol degradation in Mycobacterium tuberculosis reveal how the bacterium's metabolism is constrained by the intracellular environment. PLoS Pathog 11, e1004679.
Via, L. E., Lin, P. L., Ray, S. M., Carrillo, J., Allen, S. S., Eum, S. Y., Taylor, K., Klein, E., Manjunatha, U., Gonzales, J., et al. (2008). Tuberculous granulomas are hypoxic in guinea pigs, rabbits, and nonhuman primates. Infect Immun 76, 2333-2340.
Wang, X., Inoyama, D., Russo, R., Li, S. G., Jadhav, R., Stratton, T. P., Mittal, N., Bilotta, J. A., Singleton, E., Kim, T., et al. (2020). Antitubercular triazines: optimization and intrabacterial metabolism. Cell Chem Biol 27, 172-185.e111.
Wang, X., Perryman, A. L., Li, S. G., Paget, S. D., Stratton, T. P., Lemenze, A., Olson, A. J., Ekins, S., Kumar, P., and Freundlich, J. S. (2019). Intrabacterial metabolism obscures the successful prediction of an InhA inhibitor of Mycobacterium tuberculosis. ACS Infect Dis 5, 2148-2163.
World Health Organization. (2020). Global tuberculosis report 2020. https://apps.who.int/iris/bitstream/handle/10665/336069/9789240013131-eng.pdf (Last accessed 12 Jan. 2021).
Yates, R. M., and Russell, D. G. (2008). Real-time spectrofluorometric assays for the lumenal environment of the maturing phagosome. Methods Mol Biol 445, 311-325.
Zheng, H., Colvin, C. J., Johnson, B. K., Kirchhoff, P. D., Wilson, M., Jorgensen-Muga, K., Larsen, S. D., and Abramovitch, R. B. (2017). Inhibitors of Mycobacterium tuberculosis DosRST signaling and persistence. Nat Chem Biol 13, 218-225.

1. A compound of Formula I 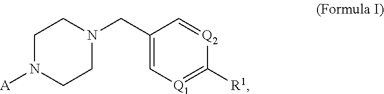
or a pharmaceutically acceptable salt thereof,
wherein—
R₁ is —SR³, —OR³, or hydrogen and R³ is selected from an unsaturated or saturated, unbranched or branched, unsubstituted or substituted C₁-C₄ alkyl and hydrogen;
Q₁ and Q₂ are independently selected from N or CH; and
A is an unsubstituted or substituted heteroaryl; and
wherein the compound is not 2-(4-((2-(ethylthio)pyrimidin-5-yl)methyl)piperazin-1-yl)benzo[d]oxazole (C6) or 6-[4-[(2-propylsulfanylpyrimidin-5-yl)methyl]piperazin-1-yl]-7H-purine (C6 analog).

2. The compound of claim 1 of Formula II 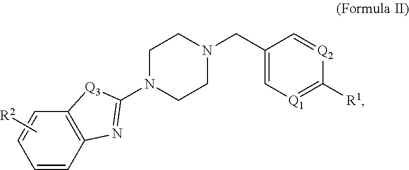
wherein—
Q₃ is O, S, or NH and
R² is selected from hydrogen, cyano, a halo, an unsaturated or saturated, unbranched or branched, unsubstituted or substituted C₁-C₄ alkoxyl; or an unsaturated or saturated, unbranched or branched, unsubstituted or substituted C₁-C₄ alkyl.

3. The compound of any one of claims 1-2, wherein R¹ is SCH₂CH₃.

4. The compound of any one of claims 1-3, wherein at least one of Q₁ or Q₂ is N.

5. The compound of any one of claims 1-4, wherein Q₃ is O.

6. The compound of claim 2, wherein the compound is selected from: 2-(4-((2-(ethylthio)pyrimidin-5-yl)methyl)piperazin-1-yl)benzo[d]thiazole (JSF-4298); 2-(4-((2-(ethylthio)pyrimidin-5-yl)methyl)piperazin-1-yl)-1H-benzo[d]imidazole (JSF-4299); 2-(4-(4-(ethylthio)benzyl)piperazin-1-yl)benzo[d]oxazole (JSF-4300); 6-chloro-2-(4-((2-(ethylthio)pyrimidin-5-yl)methyl)piperazin-1-yl)benzo[d]oxazole (JSF-4467); 2-(4-((2-(ethylthio)pyrimidin-5-yl)methyl)piperazin-1-yl)-5-methylbenzo[d]oxazole (JSF-4471); 5-chloro-2-(4-((2-(ethylthio)pyrimidin-5-yl)methyl)piperazin-1-yl)benzo[d]oxazole (JSF-4477); 2-(4-((2-(ethylthio)pyrimidin-5-yl)methyl)piperazin-1-yl)-6-(trifluoromethyl)benzo[d]oxazole (JSF-4507); 2-(4-((2-(ethylthio)pyrimidin-5-yl)methyl)piperazin-1-yl)-6-fluorobenzo[d]oxazole (JSF-4509); 2-(4-((2-(ethylthio)pyrimidin-5-yl)methyl)piperazin-1-yl)-7-fluorobenzo[d]oxazole (JSF-4516); 2-(4-((2-(ethylthio)pyrimidin-5-yl)methyl)piperazin-1-yl)benzo[d]oxazole-5-carbonitrile (JSF-4522); 2-(4-((2-(ethylthio)pyrimidin-5-yl)methyl)piperazin-1-yl)-5,6-difluorobenzo[d]oxazole (JSF-4525); 2-(4-((2-(ethylthio)pyrimidin-5-yl)methyl)piperazin-1-yl)-5-(trifluoromethyl)benzo[d]oxazole (JSF-4526); 2-(4-((2-(ethylthio)pyrimidin-5-yl)methyl)piperazin-1-yl)-6-methylbenzo[d]oxazole (JSF-4527); 2-(4-((2-(ethylthio)pyrimidin-5-yl)methyl)piperazin-1-yl)-5-methoxybenzo[d]oxazole (JSF-4528); 2-(4-((2-(ethylthio)pyrimidin-5-yl)methyl)piperazin-1-yl)-6-methoxybenzo[d]oxazole (JSF-4534); 2-(4-((2-(ethylthio)pyrimidin-5-yl)methyl)piperazin-1-yl)-5-fluorobenzo[d]oxazole (JSF-4535); 2-(4-((2-(ethylthio)pyrimidin-5-yl)methyl)piperazin-1-yl)benzo[d]oxazole-6-carbonitrile (JSF-4538); (4547); 2-(4-((2-(ethylthio)pyrimidin-5-yl)methyl)piperazin-1-yl)-4-methylbenzo[d]oxazole (JSF-4551); 2-(4-((2-(ethylthio)pyrimidin-5-yl)methyl)piperazin-1-yl)-7-methylbenzo[d]oxazole (JSF-4562); 2-(4-((2-(ethylthio)pyrimidin-5-yl)methyl)piperazin-1-yl)-7-(trifluoromethyl)benzo[d]oxazole (JSF-4601); 2-(4-((2-(ethylthio)pyrimidin-5-yl)methyl)piperazin-1-yl)-4-fluorobenzo[d]oxazole (JSF-4602); 7-chloro-2-(4-((2-(ethylthio)pyrimidin-5-yl)methyl)piperazin-1-yl)benzo[d]oxazole (JSF-4603); 4-chloro-2-(4-((2-(ethylthio)pyrimidin-5-yl)methyl)piperazin-1-yl)benzo[d]oxazole (JSF-4604); 4-chloro-2-(4-((2-(ethylthio)pyrimidin-5-yl)methyl)piperazin-1-yl)benzo[d]oxazole (JSF-4604); 2-(4-((2-(methylthio)pyrimidin-5-yl)methyl)piperazin-1-yl)benzo[d]oxazole (JSF-4730); and 2-(4-((2-(ethylthio)pyrimidin-5-yl)methyl)piperazin-1-yl)oxazole (JSF-4747).

7. The compound of claim 2, wherein the compound is 2-(4-((2-(ethylthio)pyrimidin-5-yl)methyl)piperazin-1-yl)benzo[d]oxazole-6-carbonitrile (JSF-4538).

8. The compound of any one of claims 1-7, wherein the compound inhibits growth or proliferation of Mycobacterium tuberculosis (Mtb) in a macrophage.

9. The compound of any one of claims 1-8, wherein the compound inhibits growth or proliferation of Mycobacterium tuberculosis (Mtb) in a cholesterol media.

10. A pharmaceutical composition comprising the compound according to any one of claims 1-9 and a pharmaceutically acceptable excipient, carrier, or diluent.

11. A compound of Formula (III): 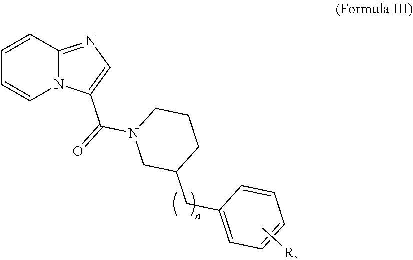
wherein n is an integer that equals to 0, 1, or 2;
wherein the phenyl group is optionally substituted with one or more R substituents independently selected from a halo, a halo substituted alkyl, a halo substituted alkoxy, or nitrile;
with the proviso that when n equals to 2, the phenyl group is not substituted at the meta position with a trifluoromethyl group.

12. The compound of claim 11, wherein the phenyl group is substituted with one R substituent.

13. The compound of claim 11, wherein the phenyl group is substituted with two R substituents.

14. The compound of any one of claims 12-13, wherein the halo is a fluoro or a chloro.

15. The compound of any one of claims 12-13, wherein the halo substituted alkyl is a trifluoromethyl.

16. The compound of any one of claims 12-13, wherein the halo substituted alkoxy is a trifluoromethoxy.

17. A pharmaceutical composition comprising the compound according to any one of claims 11-16 and a pharmaceutically acceptable excipient, carrier, or diluent.

18. A method for the treatment of a subject in need of a treatment for an infection by a microbe, the method comprising administering an effective amount of a compound or a pharmaceutical composition comprising the effective amount of a compound to the subject, wherein the compound is a chloride-response modulator.

19. The method of claim 18, wherein the microbe is Mycobacterium tuberculosis (Mtb).

20. The method of any one of claims 18-19, wherein growth or proliferation of the microbe is inhibited in the lungs of the subject.

21. The method of any one of claims 18-20, wherein the compound accumulates in a macrophage.

22. The method of any one of claims 18-21, wherein the compound accumulates in the microbe.

23. A method for inhibiting growth or proliferation of a microbe in a host cell, the method comprising contacting the host cell with an effective amount of a compound, wherein the compound is a chloride-response modulator.

24. The method of claim 23, wherein the host cell is a macrophage.

25. The method of any one of claims 23-24, wherein the compound accumulates in the host cell.

26. The method of any one of claims 23-25, wherein the compound accumulates in the microbe.

27. The method of any one of claims 23-26, wherein growth or proliferation of the microbe in the host cell is inhibited in a subject in need of a treatment for an infection by the microbe.

28. The method of any one of claims 23-27, wherein the microbe is Mycobacterium tuberculosis (Mtb).

29. The method according to any one of claims 18-28, wherein the compound is the compound according to any one of claims 1-9.

30. The method according to any one of claims 18-28, wherein the compound is the compound is 2-(4-((2-(ethylthio)pyrimidin-5-yl)methyl)piperazin-1-yl)benzo[d]oxazole (C6) or 6-[4-[(2-propylsulfanylpyrimidin-5-yl)methyl]piperazin-1-yl]-7H-purine (C6 analog).

31. The method according to any one of claims 18-28, wherein the compound is the compound according to any one of claims 11-16.

32. The method according to any one of claims 18-28, wherein the compound is imidazo[1,2-a]pyridin-3-yl(3-(3-(trifluoromethyl)phenethyl)piperidin-1-yl)methanone (C5) or pyrazolo[1,5-a]pyridin-3-yl-[3-[2-[3-(trifluoromethyl)phenyl]ethyl]piperidin-1-yl]methanone (C5 analog).
